(19) United States
(12) Patent Application Publication
(10) Pub. No.: US 2022/0162587 A1
(43) Pub. Date: May 26, 2022

(54) RNA STABILIZATION

(71) Applicant: Team Medical LLC, Boulder, CO (US)

(72) Inventors: Kyle P. Heim, Boulder, CO (US); Warren P. Heim, Boulder, CO (US)

(21) Appl. No.: 17/531,297

(22) Filed: Nov. 19, 2021

Related U.S. Application Data
(60) Provisional application No. 63/116,602, filed on Nov. 20, 2020.

(51) Int. Cl. C12N 15/10; C12N 15/113

(52) U.S. Cl. CPC C12N 15/1024; C12N 15/113

(57) ABSTRACT

Formulations of substances comprising at least one RNA stabilizing substance and at least one substance comprising RNA or based on RNA and methods of using the formulations to improve the storage and use stability of substances comprising RNA or based on RNA.

### CROSS REFERENCE TO RELATED APPLICATIONS

This application claims priority to U.S. Provisional Application No. 63/116,602 filed Nov. 20, 2021, the entire contents of which are incorporated herein by reference.

### FIELD OF THE INVENTION

The present invention relates to formulations and methods of using the formulations to improve the stability of various types of extracellular RNA and substances based on various types of extracellular RNA for storage and use in non-clinical applications and clinical applications including laboratory applications and for therapeutic uses to diagnose the health or improve the health of living organisms including plants and animals including treating humans including diagnosis of diseases and treatment of diseases or other adverse health effects in animals including in humans.

### BACKGROUND OF THE INVENTION

Ribonucleic acid (RNA) is responsible for the transcription of the genetic information stored in deoxyribonucleic acid (DNA) in a form that can be used in cells to synthesize proteins. The use of therapeutic RNA to beneficially produce proteins, regulate gene expression, or induce immune responses to specific antigens and biomarkers has become an emerging part of the field of nucleic acid therapy. The potential applications and recognized advantages of RNA based nucleic acid therapies continue to increase. For example, the recent COVID-19 infections in humans have led to vaccines developed using messenger RNA (mRNA). RNA therapies, such as vaccines using mRNA have advantages compared to other therapies, such as traditional vaccines that use inactivated, attenuated, or genetically modified microorganisms, purified antigens, or viral vectors. These other therapies can lead to adverse reactions, side effects, allergic reactions, or can develop mutations, either during manufacturing or administration, that can alter the efficacy or lead to safety concerns. RNA encodes the genetic information for the target therapy to be produced endogenously within the host cell without the need to synthesize and purify individual antigens, thereby creating greater flexibility to specifically tailor different therapies for a variety of diseases and simplifying the manufacturing process by allowing the target cells to facilitate the production of the necessary proteins and reduce or eliminate the complications of traditional vaccines.

Using RNA for nucleic acid therapies has distinct advantages compared to DNA based therapies due to the relatively short half-life of RNA and the transient message encoded within the RNA that does not require entry into the nucleus for proper expression necessary to carry out the desired function. Furthermore, DNA can potentially integrate into the host cell genome and alter genomic DNA or also become inherited by progeny. However, the investigation of uses of RNA and the production of products using RNA is complicated by the limited stability of RNA. RNA is not as stable as DNA due to RNA's single stranded nature in many biological systems and the substitution of ribose within the sugar phosphate backbone (instead of deoxyribose in DNA), leading to the presence of a 2′-hydroxyl group within the structure of the RNA backbone. The single stranded nature of RNA and the presence of the 2′-hydroxyl makes RNA susceptible to hydrolysis, in which the RNA molecule is cleaved by breaking the phosphodiester bond in the sugar-phosphate backbone, leading to degradation of the RNA molecule. Storing RNA, including storing mRNA-based vaccines, requires conditions that slow or prevent RNA from degrading, as described below, and interfering with the desired effects that RNA induces in targeted living cells.

Storage at extreme low temperatures below the freezing point of water, such as at or below about −20° C. or even at or below about −80° C., is known to be useful or even required for durable storage of RNA, including, but not limited to, for example, vaccines based on mRNA.

Storing at extreme low temperatures is more complicated than storing at more easily achieved temperatures such as temperatures approximately at the freezing point of water or refrigerated temperatures or even room temperatures or other ambient temperatures. Among the complications associated with temperatures below ambient temperatures are that refrigeration means are needed. Such refrigeration means includes cooling using thermodynamic cycles such as mechanical refrigeration (including freezers and refrigerators), frozen carbon dioxide (dry ice), or frozen water (ice).

Storing at extreme low temperatures such as at or below about −70° C. or even at or below about −80° C. or at or below about −20° C. or at or below about 4° C. complicates and adds expense to storing substances containing or based on RNA, including the storage, transportation, and therapeutic access for administration of vaccines. The complexity of using RNA is reduced the closer to room temperatures or other ambient temperatures that RNA substances can be stable.

To reduce the complexity of storing RNA substances, including mRNA and substances containing or based on mRNA such as mRNA vaccines or other therapeutic products, materials and methods for improving the stability of RNA substances so that storage can be done at temperatures greater than extremely cold temperatures are needed.

Lyophilization or freeze-drying RNA substances is used to improve the storage stability of RNA and reduce the need for storing RNA at cold temperatures or even extreme cold temperatures. However, freeze-drying and lyophilization requires specialized equipment and adds significant time, expense, and complexity to the production and storage of RNA, including but not limited to mRNA and substances containing or based on mRNA such as mRNA vaccines or other therapeutic products.

Encapsulating or complexing RNA substances with nanoparticles or lipid nanoparticles is used to improve the delivery of an RNA substance to a cell or tissue. Nanoparticles may incorporate polyethylene glycol (PEG) modified lipids, PEG conjugated lipids, or similar modifications to improve nanoparticle stability. These modifications improve the stability of the nanoparticle by decreasing aggregation and agglomeration, as well as reducing protein binding and opsonization. However, improving nanoparticle stability relates to maintaining consistent nanoparticle size and distribution as well as improving circulation half-life and reducing systemic clearance of nanoparticles following administration of the encapsulated or complexed RNA and does not necessarily improve RNA stability by preventing RNA degradation during storage or shipping or reducing the need for storing or shipping RNA at cold temperatures or even extreme cold temperatures.

Aprotic substances used during nucleic acid or RNA synthesis are not necessarily used to improve RNA stability by preventing RNA degradation during storage or shipping and are not necessarily used to reduce the need for storing or shipping RNA at cold temperatures or even extreme cold temperatures following synthesis. Instead of being used to improve RNA stability during storage or shipping by reducing RNA degradation, aprotic substances, including DMSO, are often used during PCR or DNA or RNA synthesis to denature or decrease DNA or RNA duplex stability and disrupt or alter secondary structure of RNA or DNA to help facilitate synthesis of GC rich regions or GC rich templates and denature double stranded nucleic acid or reduce sequence variability associated with the efficiency of nucleic acid synthesis. These aprotic substances, such as DMSO, help reduce the melting temperature (Tm) of nucleic acid secondary structure or base paring or reduce the Tm needed to denature double stranded nucleic acid, allowing enzymes involved in transcription or nucleic acid synthesis to efficiently read the template nucleic acid strand. The use of aprotic substances in the context of reducing or altering secondary structure or duplex stability (including denaturing double stranded nucleic acid) during nucleic acid synthesis relates to improving the efficiency of synthesis by disrupting base-pairing interactions, reducing or altering DNA or RNA secondary structure or duplex stability and are not used for the purpose of preventing or reducing RNA degradation during storage or shipping. Furthermore, following the use of aprotic substances to reduce or alter the secondary structure and improve efficiency of nucleic acid synthesis, the synthesized nucleic acids are purified following the synthesis and the aprotic substances used during synthesis are removed.

### SUMMARY OF THE INVENTION

Accordingly, a primary objective of the present invention is to provide substances for storage environments for RNA substances and methods using substances in storage environments for RNA substances that reduce degradation of RNA substances so that RNA substances have improved stability when stored at temperatures above about −80° C. As used herein, the terms RNA substance or RNA substances means a substance or substances comprised of at least one of extracellular RNA or purified extracellular RNA and may include but is not limited to, mRNA and vaccines, therapeutics, or medicaments, based on mRNA. As used herein, the term storage environment means the substances in which one or more RNA substance is present other than when being synthesized or transcribed or deployed for immediate use.

Another primary objective of the present invention is to provide substances for storage environments for RNA substances and methods using substances in storage environments for RNA substances that reduces degradation of RNA substances so that RNA substances have improved stability when stored at temperatures at or above about −20° C. Another primary objective of the present invention is to provide substances for storage environments and methods using substances in storage environments for RNA substances that reduces degradation of RNA substances so that RNA substances have improved stability when stored at temperatures at or above about 4° C. Another primary objective of the present invention is to provide substances for storage environments and methods using substances in storage environments for RNA substances that reduces degradation of RNA substances so that RNA substances have improved stability when stored at temperatures at or above about 20° C.

Another primary objective of the present invention is to provide storage environments for RNA substances that reduce the needs for storing, transporting, distributing, or storing at a point of use such as a point for therapeutic administration (collectively “storage” or “storage and use” hereinafter) using thermodynamic cycle cooling such as vapor compression mechanical cooling or absorption cooling. Another primary objective of the present invention is to provide storage environments for RNA substances that reduce the needs for storage and use of RNA substances using dry ice. Another primary objective of the present invention is to provide storage environments for RNA substances that reduce the needs for storage and use of RNA substances using ice. Another primary objective of the present invention is to provide storage environments for RNA substances that reduce the needs for storage and use of RNA substances at temperatures of about −80° C. Another primary objective of the present invention is to provide storage environments for RNA substances that reduce the needs for storage and use of RNA substances at temperatures of about −20° C. Another primary objective of the present invention is to provide storage environments for RNA substances that reduce the needs for storage and use of RNA substances at temperatures of about 4° C.

Another primary objective of the present invention is to provide delivery substances for nucleic acid, including RNA substances, including but not limited to mRNA and vaccines, therapeutics, or medicaments based on mRNA, to cells that stabilize RNA and reduce degradation of the RNA to simplify RNA delivery, shipping, manufacturing, or storage.

The storage environment for RNA substances includes an environment that contains at least one or more RNA stabilizing substances that is combined with at least one RNA substance such as by mixing, pipetting, blending, submerging, vortexing, shaking, lyophilizing, vaporizing, or sublimating such that the RNA stabilizing substance is at least intimately associated with or partially contacting or at least partially encapsulating the RNA substance. The storage environment may include at least one RNA stabilizing substance and at least one RNA substance that are stored separately with the intention of combining or mixing the RNA substance with the RNA stabilizing substance either prior to or during use of the RNA substance. The storage environment includes the immediate environment of the RNA substance such as occurs when the RNA substance is mixed or is otherwise in close association or at least partially or substantially contacting one or more RNA stabilizing substances. Dimethyl sulfoxide (DMSO) is an example RNA stabilizing substance that may be used. As a non-limiting example, the storage environment for RNA substances may be at least some DMSO intimately contacting at least part of one or more RNA substances at the molecular level such as may result from submerging, blending, or mixing one or more RNA substances with the aprotic substance DMSO.

The inventors have discovered that aprotic substances, described in detail elsewhere herein, may be RNA stabilizing substances. In a non-limiting example, an RNA stabilizing substance may be an aprotic substance such as, DMSO.

The inventors have discovered that polar aprotic substances, described in detail elsewhere herein, may be RNA stabilizing substances. In a non-limiting example, an RNA stabilizing substance may be a polar aprotic substance such as, DMSO.

The inventors have discovered that mixtures comprising one or more RNA substance and at least one RNA stabilizing substance improves RNA stability. The inventors have also discovered that RNA stability may be improved with mixtures comprising one or more RNA substance and two or more RNA stabilizing substances. The inventors have also discovered that RNA stability may be improved with mixtures comprising one or more RNA substance and three or more RNA stabilizing substances.

As used herein, the terms stabilize RNA or stabilizing RNA means reducing degradation of RNA substances.

As used herein, an RNA stabilizing substance or stabilizing RNA substance means a substance that stabilizes RNA of at least one or more RNA substance. RNA stabilizing substances provide an environment for the RNA substance that makes the RNA substance at least as stable at a higher temperature than the stability the RNA substance would have at a lower temperature.

As used herein, cells means in vivo, in vitro, in situ, or ex vivo cells, including but not limited to eukaryotic cells, prokaryotic cells, plant cells, fungal cells, insect cells, bacterial cells, mycoplasma, protozoa, plasmodium, or mammalian cells, including but not limited to the cells of vertebrate animals and the cells of humans.

As used herein, nucleic acid means DNA, RNA, polymeric, single-stranded, double-stranded, or more highly aggregated hybridization motifs, and may include chemical modifications, derivatives, or analogs thereof. Modifications may include but are not limited to modifications comprising backbone modifications, sugar modifications, or base modifications. Modifications may also include, but are not limited to, those providing chemical groups that incorporate additional charge, polarizability, hydrogen bonding, electrostatic interaction, points of attachment or functionality to the nucleic acid bases or to the nucleic acid as a whole. Such modifications may include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine or isoguanidine or the like. Nucleic acids may also include non-natural bases, such as, for example, nitroindole. Modifications may also include 3′ modifications or 5′ modifications such as capping with a fluorophore (e.g., quantum dot) or another moiety. Nucleotides, may be referred to by their commonly accepted single-letter codes.

As used herein, the term RNA means ribonucleic acid, and may include chemical modifications, derivatives, or analogs thereof, with the exception of a chemical modification rendering the RNA into DNA. RNA may include RNA analogs, including but not limited to synthetic RNA analogs or nucleotide analogs. RNA may also include non-natural synthetic ribonucleotides. The RNA may be polymeric, single-stranded, or double stranded, or a more highly aggregated form. The RNA may be provided by any means known in the art, including but not limited to, in vitro transcription, purification from an organism, chemical synthesis, or a combination thereof. The RNA may be, but is not limited to, mRNA, rRNA, tRNA, microRNA, small interfering RNA (siRNA), self-amplifying RNA, small activating RNA, tmRNA, dsRNA, shRNA, snRNA, antisense RNA (asRNA), eRNA, RNA enzymes, CRISPR RNA, or total RNA. The RNA may be purified RNA (e.g., purified mRNA, purified rRNA, purified tRNA, purified microRNA, purified siRNA, purified self-amplifying RNA, purified small activating RNA, purified tmRNA, purified dsRNA, purified shRNA, purified snRNA, purified asRNA, purified eRNA, purified RNA enzymes, purified CRISPR RNA, or purified total RNA). Furthermore, RNA modifications may include but are not limited to modifications comprising backbone modifications, sugar modifications, or base modifications. RNA modifications may also include, but are not limited to, 5′ modifications or 3′ modifications, and may also include, but are not limited to, 5′-cap, 5′-cap structures, 5′-cap modifications, or 5′-cap analogs. RNA modifications may also include, but are not limited to, lipid modifications or PEG modifications, wherein a lipid or polyethylene glycol may be attached or covalently linked to an RNA molecule.

As used herein, the terms RNA substance or RNA substances means a substance or substances comprised of at least one of extracellular RNA or purified extracellular RNA. RNA substance or RNA substances may include, but are not limited to, substances comprising one or more polymeric forms of RNA, including but not limited to single stranded or multiple stranded (including double stranded) forms that may include, but are not limited to, coding or non-coding forms of RNA. RNA substance or RNA substances may also include, but are not limited to, mRNA and vaccines, therapeutics, diagnostics, or medicaments based on RNA, mRNA, or sections of RNA or other forms of ribonucleic acid that may be used for, including but not limited to, therapeutic, diagnostic, analysis, in vitro, in vivo, ex vivo, in situ, delivery, manufacturing, storage or other purposes. RNA substance or RNA substances may include, but are not limited to, mRNA, self-amplifying RNA, small activating RNA, rRNA, tRNA, microRNA, siRNA, tmRNA, dsRNA, shRNA, snRNA, asRNA, eRNA, RNA enzymes, CRISPR RNA, or total RNA. RNA substance or RNA substances may include, but are not limited to, purified RNA, including but not limited to, purified mRNA, purified rRNA, purified tRNA, purified microRNA, purified siRNA, purified self-amplifying RNA, purified small activating RNA, purified tmRNA, purified dsRNA, purified shRNA, purified snRNA, purified asRNA, purified eRNA, purified RNA enzymes, purified CRISPR RNA, or purified total RNA.

RNA modifications of the present invention may include, but are not limited to, those described in US Patent Application Pub. No. US 2020/0383922, incorporated herein by reference, as RNA modifications, chemical modifications, backbone modifications, sugar modifications, base modifications, nucleotide analogues/modifications, modified nucleosides, nucleoside modifications, lipid modifications, or 5′-CAP structures.

RNA modifications of the present invention may include, but are not limited to, those described in U.S. Pat. No. 10,702,600, incorporated herein by reference, as chemical modifications, modifications of polynucleotides, modified RNA polynucleotides, nucleoside or nucleotide modifications, modified nucleobases, or naturally occurring or non-naturally occurring modifications.

RNA modifications of the present invention may include, but are not limited to, those described in PCT Patent Application Pub. No. WO 2021/156267 A1, incorporated herein by reference, as 5′-cap structures, cap analogues, RNA modifications, modified RNA, chemical modifications, backbone modifications, sugar modifications, base modifications, nucleotide analogues/modifications, or modified nucleotides.

RNA modifications of the present invention may include, but are not limited to, those described in US Patent Application Pub. No. US 2021/0260097, incorporated herein by reference, as modified mRNAs, mmRNAs, modified nucleobases, modified nucleosides, modified nucleotides, chemically modified mRNAs, or nucleoside modifications.

RNA modifications of the present invention may include, but are not limited to, those described in PCT Patent Application Pub. No. WO 2021/213945 A1, incorporated herein by reference, as modified nucleosides, further modified nucleosides, modified nucleobases, modified nucleotides, 5′-cap, 5′cap-analog, or capping structure at the 5′-end of the RNA.

In one embodiment one or more RNA substance may comprise an open reading frame. In one embodiment one or more RNA substance may not comprise an open reading frame. In one embodiment one or more RNA substance may comprise a 5′-cap or 5′-cap structure. In one embodiment one or more RNA substance may comprise a 5′ UTR. In one embodiment one or more RNA substance may comprise a 3′ UTR. In one embodiment one or more RNA substance may comprise a poly (A)-tail.

In one embodiment one or more RNA substance may be polymeric. In one embodiment one or more RNA substance may be single stranded. In one embodiment one or more RNA substance may be double stranded. In one embodiment one or more RNA substance may have one or more complimentary strands or partially complimentary strands, wherein a complimentary or partially complementary strand may include, but is not limited to, an RNA strand, DNA strand, peptide nucleic acid strand or other type of complementary or partially complementary strand.

In one embodiment one or more RNA substance may comprise a coding RNA. As a non-limiting example, a coding RNA may include, but is not limited to, mRNA or self-amplifying RNA.

In one embodiment one or more RNA substance may comprise a non-coding RNA. As a non-limiting example, a non-coding RNA may include, but is not limited to, microRNA, siRNA, CRISPR RNA, antisense RNA, small activating RNA, or RNA enzymes.

In one embodiment an RNA substance may be comprised of 2-1,000,000 nucleotides. In one embodiment an RNA substance may be comprised of 2-500,000 nucleotides. In one embodiment an RNA substance may be comprised of 2-100,000 nucleotides. In one embodiment an RNA substance may be comprised of 2-50,000 nucleotides. In one embodiment an RNA substance may be comprised of 2-20,000 nucleotides. In one embodiment an RNA substance may be comprised of 2-10,000 nucleotides.

In one embodiment an RNA substance may be comprised of 5-1,000,000 nucleotides. In one embodiment an RNA substance may be comprised of 5-500,000 nucleotides. In one embodiment an RNA substance may be comprised of 5-100,000 nucleotides. In one embodiment an RNA substance may be comprised of 5-50,000 nucleotides. In one embodiment an RNA substance may be comprised of 5-20,000 nucleotides. In one embodiment an RNA substance may be comprised of 5-10,000 nucleotides.

In one embodiment an RNA substance may be comprised of 10-1,000,000 nucleotides. In one embodiment an RNA substance may be comprised of 10-500,000 nucleotides. In one embodiment an RNA substance may be comprised of 10-100,000 nucleotides. In one embodiment an RNA substance may be comprised of 10-50,000 nucleotides. In one embodiment an RNA substance may be comprised of 10-20,000 nucleotides. In one embodiment an RNA substance may be comprised of 10-10,000 nucleotides.

In one embodiment an RNA substance may be comprised of 20-1,000,000 nucleotides. In one embodiment an RNA substance may be comprised of 20-500,000 nucleotides. In one embodiment an RNA substance may be comprised of 20-100,000 nucleotides. In one embodiment an RNA substance may be comprised of 20-50,000 nucleotides. In one embodiment an RNA substance may be comprised of 20-20,000 nucleotides. In one embodiment an RNA substance may be comprised of 20-10,000 nucleotides.

In one embodiment an RNA substance may be comprised of 100-1,000,000 nucleotides. In one embodiment an RNA substance may be comprised of 100-500,000 nucleotides. In one embodiment an RNA substance may be comprised of 100-100,000 nucleotides. In one embodiment an RNA substance may be comprised of 100-50,000 nucleotides. In one embodiment an RNA substance may be comprised of 100-20,000 nucleotides. In one embodiment an RNA substance may be comprised of 100-10,000 nucleotides.

Descriptions of substances herein may include one or more forms of the substance. These forms may include, but are not limited to, isomers, structural isomers, stereo isomers, chiral forms, salts, or combinations thereof.

As used herein, organic or organic substance means a substance comprised of at least one or more carbon atom, wherein at least one or more carbon atom is covalently bonded to at least one or more hydrogen atom.

As used herein, an aprotic substance is an organic substance that is substantially incapable of donating or accepting hydrogen ions at one or more pH in the range of about physiologic pH. Therefore, an aprotic substance generally does not contribute to hydrogen ion exchange at one or more pH in the range of about physiologic pH.

As used herein, a polar aprotic substance is an aprotic substance comprised of at least one oxygen atom or at least one nitrogen atom.

As used herein, physiologic pH means pH in the range of about 5-9.

As used herein a combination of one or more RNA substances with one or more RNA stabilizing substances comprises one or more RNA substances and one or more RNA stabilizing substances and the combination may include one or more additional other substances. As a non-limiting example, a combination of at least one aprotic substance and at least one RNA substance comprises at least one aprotic substance and at least one RNA substance and other substances may also be parts of the combination.

As used herein a combination or mixture of substances is a composition that comprises the substances described.

### BRIEF DESCRIPTION OF THE DRAWINGS

For a more complete understanding of the present invention, and further advantages thereof, reference is now made to the following detailed description, taken in conjunction with the drawings, as described below.

FIG. 1 shows purified RNA following in vitro transcription analyzed by denaturing agarose gel electrophoresis. The PCR marker used is a double stranded DNA marker. RNA runs on denaturing agarose gel electrophoresis with an apparent molecular weight between approximately 1,000-1,500 bases. A black and white image of purified RNA as well as a grayscale image of purified RNA are shown.

FIG. 2 is a black and white image showing comparison of RNA stability when stored in DMSO or Tris Acetate EDTA (TAE) buffer over the course of 200 days at various temperatures ranging from room temperature (RT, approximately 20-25° C.), 4° C., −20° C., and −80° C. in accordance with the present invention. RNA degradation is indicated by changes in RNA apparent molecular weight, band sharpness, and band fluorescence intensity following denaturing agarose gel electrophoresis. Full length RNA has an apparent molecular weight between approximately 1,000-1,500 bases.

FIG. 3 is a grayscale image showing comparison of RNA stability when stored in DMSO or Tris Acetate EDTA (TAE) buffer over the course of 200 days at various temperatures ranging from room temperature (RT, approximately 20-25° C.), 4° C., −20° C., and −80° C. in accordance with the present invention. RNA degradation is indicated by changes in RNA apparent molecular weight, band sharpness, and band fluorescence intensity following denaturing agarose gel electrophoresis. Full length RNA has an apparent molecular weight between approximately 1,000-1,500 bases.

FIG. 4 shows a comparison of RNA stability when stored in various concentrations of DMSO and 50 mM sodium acetate, pH 5.2, over the course of 72 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between approximately 1,000-1,500 bases. A black and white image and a grayscale image are shown.

FIG. 5 shows a comparison of RNA stability when stored in either 1) 50 mM sodium acetate, pH 5.2; 2) 70% DMSO and 50 mM sodium acetate, pH 5.2; or 3) 70% DMSO, 500 mM acetylcholine chloride, and 50 mM sodium acetate, pH 5.2; over the course of 72 hrs at 60° C. in accordance with the present invention. RNA degradation is indicated by changes in RNA apparent molecular weight, band sharpness, and band fluorescence intensity following denaturing agarose gel electrophoresis. Full length RNA has an apparent molecular weight between approximately 1,000-1,500 bases. A black and white image and a grayscale image are shown.

FIG. 6 shows a comparison of RNA stability when stored in either 1) 50 mM sodium acetate, pH 5.2; 2) 70% DMSO and 50 mM sodium acetate, pH 5.2; 3) 70% DMSO, 1M acetylcholine chloride, and 50 mM sodium acetate, pH 5.2; or 4) 70% DMSO, 500 mM acetylcholine chloride, and 50 mM sodium acetate, pH 5.2; over the course of 72 hrs at 60° C. in accordance with the present invention. RNA degradation is indicated by changes in RNA apparent molecular weight, band sharpness, and band fluorescence intensity following denaturing agarose gel electrophoresis. Full length RNA has an apparent molecular weight between approximately 1,000-1,500 bases. A black and white image and a grayscale image are shown.

FIG. 7 shows a multi-chamber syringe loaded with components of an RNA composition in accordance with the present invention.

FIG. 8 shows an RNA composition kit in accordance with the present invention.

FIG. 9 shows an RNA composition kit in accordance with the present invention

FIG. 10 shows an RNA composition kit in accordance with the present invention

FIG. 11 shows an RNA storage package in accordance with the present invention

FIG. 12 is a flow chart illustrating a process for producing and using an RNA product in accordance with the present invention.

FIG. 13 is a flow chart illustrating a process for producing and using an RNA product in accordance with the present invention.

### DETAILED DESCRIPTION OF THE INVENTION

Before explaining various embodiments of RNA stabilizing substances, storage environments of RNA substances, and cellular delivery of RNA substances in detail, it should be noted that the illustrative embodiments and examples are not limited in application or use to the details of construction and arrangement of parts illustrated in the accompanying drawings and description. The illustrative embodiments and examples may be implemented or incorporated in other embodiments, variations and modifications, and may be practiced or carried out in various ways. Further, unless otherwise indicated, the terms and expressions employed herein have been chosen for the purpose of describing the illustrative embodiments for the convenience of the reader and are not for the purpose of limitation thereof. Also, it will be appreciated that one or more of the following-described embodiments, expressions of embodiments and/or examples, can be combined with one or more of the other following-described embodiments, expressions of embodiments and/or examples.

As used herein the term “or” means and/or unless the context states otherwise.

As used herein the term “a” means one or more unless the context states otherwise.

As used herein the term “an” means one or more unless the context states otherwise.

It is of great interest to the field of therapeutics, diagnostics, reagents, agriculture, and biological assays to increase the stability of RNA substances and reduce RNA degradation of RNA substances. Described herein, are compositions (including pharmaceutical compositions and therapeutic compositions) and methods for the design, preparation, manufacture and/or formulation of storage environments to stabilize RNA substances. Also provided are systems, processes, and devices for selection, design, and/or utilization of the storage environments to stabilize RNA described herein.

RNA is naturally unstable, including in aqueous solutions. RNA is susceptible to degradation including, but not limited to the following types of degradation: enzymatic, autocatalytic, metal-catalyzed, autohydrolysis, hydrolysis, temperature induced, pH induced, chemically induced, oxidation induced, reduction induced, or radiation induced. Without being bound to any particular mechanism or mode of action, it is believed that the present invention provides an environment that increases stability of RNA substances at warmer than extreme cold conditions by reducing the exposure to substances that may induce RNA degradation.

The present inventors have discovered that RNA stabilizing substances can surprisingly increase the stability of RNA substances at temperatures above about −80° C. The present inventors have also discovered that RNA stabilizing substances can surprisingly increase the stability of RNA substances at temperatures above about −20° C. The present inventors have also discovered that RNA stabilizing substances can surprisingly increase the stability of RNA substances at temperatures above about 4° C. The present inventors have also discovered that RNA stabilizing substances can surprisingly increase the stability of RNA substances at temperatures above about 10° C. The present inventors have also discovered that RNA stabilizing substances can surprisingly increase the stability of RNA substances at temperatures above about 20° C.

The present inventors have discovered that combinations of RNA stabilizing substances in mixtures containing at least one RNA substance lead to increases in RNA stability compared to mixtures containing at least one RNA substance without all components of the combination of RNA stabilizing substances.

Embodiments of the present invention may comprise one or more RNA stabilizing substances.

Embodiments of the present invention may include compositions comprising a combination of one or more RNA stabilizing substance with one or more nucleic acid substance.

Embodiments of the present invention may include compositions comprising a combination of one or more RNA stabilizing substance with one or more DNA substance.

Embodiments of the present invention may include compositions comprising a combination of one or more RNA stabilizing substance with one or more RNA substance.

An embodiment of the present invention includes a combination of materials that comprises at least one or more RNA substance and at least one or more RNA stabilizing substance. Another embodiment of the present invention includes a combination of materials that comprises at least one RNA substance and a mixture of RNA stabilizing substances in which the mixture of RNA stabilizing substances comprises at least two RNA stabilizing substances. Another embodiment of the present invention includes a combination of materials that comprises at least one RNA substance and a mixture of RNA stabilizing substances in which the mixture of RNA stabilizing substances comprises at least three RNA stabilizing substances. Another embodiment of the present invention includes a combination of materials that comprises at least one RNA substance and a mixture of RNA stabilizing substances in which the mixture of RNA stabilizing substances comprises at least four RNA stabilizing substances. Another embodiment of the present invention includes a combination of materials that comprises at least one RNA substance and a mixture of RNA stabilizing substances in which the mixture of RNA stabilizing substances comprises at least five RNA stabilizing substances.

An embodiment of the present invention includes a combination of materials that comprises at least one or more RNA substance and at least one or more RNA stabilizing substance that improves RNA stability. The inventors have discovered that RNA stability may be improved with mixtures comprising at least one or more RNA substance and multiple RNA stabilizing substances in which the number of multiple RNA stabilizing substances is preferably between two and five. As a non-limiting example, RNA stability may be improved with mixtures comprising at least one or more RNA substance and multiple RNA stabilizing substances where the number of multiple RNA stabilizing substances is five.

The environment that improves the stability of RNA substances may comprise at least one or more vapor, liquid, powder, or solid RNA stabilizing substance.

As described herein, the present inventors have discovered that selected materials may be used individually as RNA stabilizing substances and that selected materials may be used in combinations as RNA stabilizing substances.

The inventors have discovered previously unrecognized substances stabilize RNA.

The inventors have discovered that aprotic substances may be RNA stabilizing substances. The inventors have also discovered that polar aprotic substances may be RNA stabilizing substances. The inventors have also discovered that aprotic choline-based esters may be RNA stabilizing substances.

Embodiments of the present invention may comprise one or more RNA stabilizing substance. Other embodiments of the present invention may comprise one or more RNA substance and one or more RNA stabilizing substance.

The inventors have discovered that RNA stabilizing substances may stabilize RNA substances.

The inventors have also discovered that the stability of RNA substances may be enhanced in an RNA storage environment comprising one or more RNA stabilizing substance.

The inventors have also discovered that the stability of RNA substances may be enhanced in compositions comprising an RNA substance and one or more RNA stabilizing substance.

One embodiment of the present invention includes a composition comprising a combination of one or more RNA substance and one or more RNA stabilizing substance.

One embodiment of the present invention includes a combination comprising a mixture of one or more RNA substance and one or more RNA stabilizing substance.

Embodiments of the present invention that comprise one or more RNA substance and one or more RNA stabilizing substance may include combining, such as by mixing, one or more RNA substance with one or more substance that comprises at least one or more RNA stabilizing substance.

An embodiment of the present invention includes compositions of materials that comprise one or more RNA substance and at least one or more RNA stabilizing substance. The environment that improves the stability of RNA substances may be at least one or more vapor, liquid, powder, or solid RNA stabilizing substance.

An embodiment of the present invention includes combinations of materials that comprise one or more RNA substance and at least one or more RNA stabilizing substance. The environment that improves the stability of RNA substances may be at least one or more vapor, liquid, powder, or solid RNA stabilizing substance.

In one embodiment of the present invention a composition comprising a combination of one or more RNA substance and one or more RNA stabilizing substance, produces a mixture with at least one or more RNA substance and at least one or more RNA stabilizing substance.

In one embodiment of the present invention a combination comprising one or more RNA substance and one or more RNA stabilizing substance, produces a mixture with at least one or more RNA substance and at least one or more RNA stabilizing substance.

In one embodiment of the present invention a composition with improved RNA stability may comprise a combination of one or more RNA substance and one or more RNA stabilizing substance.

In one embodiment of the present invention a composition with improved RNA stability may comprise a mixture of one or more RNA substance and one or more RNA stabilizing substance.

In one embodiment of the present invention, each component included in a composition comprising one or more RNA substance and one or more RNA stabilizing substance, may be stored separately, such as in a kit, or such as individual substances or as mixtures of one or more substance, and then combined later to produce a composition comprising one or more RNA substance and one or more RNA stabilizing substance.

In one embodiment of the present invention a composition comprising one or more RNA substance and one or more RNA stabilizing substance may be at least partially biocompatible.

In one embodiment of the present invention comprising a combination of one or more RNA substance and one or more RNA stabilizing substance may be at least partially biocompatible.

Embodiments of the present invention may comprise one or more aprotic substance. Other embodiments of the present invention may comprise one or more RNA substance and one or more aprotic substance.

RNA stabilizing substances improve the stability of RNA substances in the presence of water. Embodiments of the present invention may be compositions that comprise at least one RNA stabilizing substance, at least one RNA substance, and water. These embodiments comprising water may be any composition as described herein that may also comprise water.

Embodiments of the present invention may comprise any composition described herein and may include one or more additional other substances of which water may be one of the additional other substances.

Embodiments of the present invention may comprise any composition described herein and one or more cellular uptake agent, as described herein. Such embodiments may be used as at least part of therapeutic substances, such as vaccines deploying mRNA to one or more living organisms (which may include humans or may include non-human animals) with at least one RNA stabilizing substance improving the stability of the therapeutic substance and at least one cellular uptake agent improving the efficacy of the therapeutic substance.

Embodiments of the present invention may comprise any composition described herein and one or more cellular uptake agent, as described herein. Embodiments of the present invention may comprise any composition described herein and may also include one or more additional other substance, such as one or more cellular uptake agent, buffering agent, salt, or chelating agent, as non-limiting examples.

### Mechanisms and Theory

Without being bound to any particular mechanism or mode of action, RNA hydrolysis can be initiated by a base removing a proton from the 2′-OH on the ribose sugar, leading to the subsequent nucleophilic attack of the 2′ oxygen on the adjacent phosphorus atom. Base catalyzed hydrolysis activates the 2′-OH by removing the proton and creating a negatively charged 2′ oxygen and promoting nucleophilic attack of the 2′ oxygen on the adjacent phosphorus atom of the phosphodiester backbone of RNA. Water's protic nature to both donate and accept protons allows water to act as both an acid and a base at about physiologic pH. Therefore, water is capable of acting as a proton acceptor and activating the 2′-OH to promote the nucleophilic attack of the 2′ oxygen on the adjacent phosphorus atom of the phosphodiester backbone of the RNA molecule to promote RNA hydrolysis.

Without being bound to any particular mechanism or mode of action, aprotic substances are substantially incapable of donating or accepting hydrogen ions at one or more pH in the range of about physiologic pH, and, thus, generally do not contribute to hydrogen ion exchange that may promote hydrolysis of RNA substances. Creating an environment with substantially less hydrogen ion exchange, such as in the presence of an aprotic substance, then the initiation of RNA hydrolysis and subsequent cleavage of the RNA molecule will be less favorable. Therefore, aprotic substances do not promote the nucleophilic attack of the 2′-OH on the adjacent phosphorus atom of the phosphodiester backbone, such as occurs in the presence of water. Thus, by making initiation of RNA hydrolysis less favorable, aprotic substances may be RNA stabilizing substances.

Without being bound to any particular mechanism or mode of action, an aprotic substance may at least reduce access to the RNA substance by materials that may promote RNA hydrolysis or degradation of the RNA substance. In a non-limiting example, an aprotic substance in the environment of the RNA substance may create an environment that excludes water from the RNA substance or reduces the concentration of water in the environment around the RNA substance. Therefore, if one or more aprotic substances substantially displace all of the water in the environment of the RNA substance then the RNA substance is substantially not exposed to water, ions, or other materials that may promote RNA hydrolysis. In another non-limiting example, an aprotic substance in the environment of the RNA substance may also create an environment that limits the molecular mobility of water, ions, or other materials and thereby limit and/or prevent the exposure of the RNA substance to water, ions, or other materials that may promote RNA hydrolysis.

Double stranded RNA substances are more stable than single stranded RNA substances. Without being bound to any particular mechanism or mode of action, the increased stability of double stranded RNA is at least partially a result of the decreased flexibility of the double stranded RNA substance which reduces the movement of the RNA substance creating a lower probability that a 2′-OH will be in close enough proximity to an adjacent phosphorus atom to perform a nucleophilic attack and initiate RNA hydrolysis. In a non-limiting example, an aprotic substance that reduces the flexibility or molecular movement of a single stranded RNA substance reduces the likelihood that a 2′-OH will be in close enough proximity to an adjacent phosphorus atom to perform a nucleophilic attack and initiate RNA hydrolysis.

Embodiments of the present invention that comprise an RNA substance and at least one aprotic substance may include combining, such as by mixing, at least one RNA substance with at least one substance that comprises at least one aprotic substance. Without being bound to any particular mechanism or mode of action, the aprotic substance at least reduces access to the RNA substance by materials that may promote hydrolysis or degradation of the RNA substance. In a non-limiting example, an aprotic substance in the environment of the RNA substance may reduce the concentration of water in the environment around the RNA substance. In another non-limiting example, if one or more aprotic substances substantially displace all of the water in the environment of the RNA substance then the RNA substance is substantially not exposed to water or other materials that may promote hydrolysis.

Embodiments of the present invention comprising at least one or more RNA stabilizing substance may include one or more forms of an RNA stabilizing substance. These forms may include, but are not limited to, isomers, structural isomers, stereo isomers, chiral forms, salts, or combinations thereof.

### Aprotic Substances:

The inventors have discovered that aprotic substances may stabilize RNA substances.

The inventors have discovered that RNA stabilizing substances may comprise aprotic substances. The inventors have also discovered that the stability of RNA substances may be enhanced in an RNA storage environment comprising one or more aprotic substance.

The inventors have also discovered that the stability of RNA substances may be enhanced in compositions comprising an RNA substance and one or more aprotic substance.

In one embodiment of the present invention, one or more RNA stabilizing substance may comprise one or more aprotic substance.

One embodiment of the present invention includes a composition comprising a combination of one or more RNA substance and one or more aprotic substance.

One embodiment of the present invention includes a combination comprising a mixture of one or more RNA substance and one or more aprotic substance.

Embodiments of the present invention that comprise one or more RNA substance and one or more aprotic substance may include combining, such as by mixing, one or more RNA substance with one or more substance that comprises at least one or more aprotic substance.

An embodiment of the present invention includes compositions of materials that comprise one or more RNA substance and at least one or more aprotic substance. The environment that improves the stability of RNA substances may be at least one or more vapor, liquid, powder, or solid aprotic substance.

An embodiment of the present invention includes combinations of materials that comprise one or more RNA substance and at least one or more aprotic substance. The environment that improves the stability of RNA substances may be at least one or more vapor, liquid, powder, or solid aprotic substance.

In one embodiment of the present invention a composition comprising a combination of one or more RNA substance and one or more aprotic substance, produces a mixture with at least one or more RNA substance and at least one or more aprotic substance.

In one embodiment of the present invention a combination comprising one or more RNA substance and one or more aprotic substance, produces a mixture with at least one or more RNA substance and at least one or more aprotic substance.

In one embodiment of the present invention a composition with improved RNA stability may comprise a combination of one or more RNA substance and one or more aprotic substance.

In one embodiment of the present invention a composition with improved RNA stability may comprise a mixture of one or more RNA substance and one or more aprotic substance.

In one embodiment of the present invention, each component included in a composition comprising one or more RNA substance and one or more aprotic substance, may be stored separately, such as in a kit, or such as individual substances or as mixtures of one or more substance, and then combined later to produce a composition comprising one or more RNA substance and one or more aprotic substance.

In one embodiment of the present invention a composition comprising one or more RNA substance and one or more aprotic substance may be at least partially biocompatible.

In one embodiment of the present invention comprising a combination of one or more RNA substance and one or more aprotic substance may be at least partially biocompatible.

Embodiments of the present invention comprising at least one or more aprotic substance may include one or more forms of the aprotic substance. These forms may include, but are not limited to, isomers, structural isomers, stereo isomers, chiral forms, salts, or combinations thereof.

In one embodiment of the present invention at least one aprotic substance comprising a combination of at least one aprotic substance and at least one RNA substance produces a mixture with at least one of the RNA substances. In one embodiment of the present invention comprising a combination of at least one aprotic substance and at least one RNA substance is at least partially biocompatible.

A non-limiting example of an aprotic substance is dimethyl sulfoxide (DMSO). One embodiment of the present invention may include combinations of substances comprising DMSO and another embodiment may also comprise at least one RNA substance.

Embodiments of compositions comprising RNA stabilizing substances, of which DMSO is a non-limiting example, may have different weight percent concentrations. For conciseness, the following list of weight percent concentrations with DMSO as an example aprotic substance is herein referred to as the RNA stabilizing substance weight percent concentration list. In one embodiment the concentration of DMSO may be at least 0.1 percent (all composition percentages herein are weight percent, unless stated otherwise) of the substances that are not RNA substances. In one embodiment the concentration of DMSO may be at least 1 percent of the substances that are not RNA substances. In another embodiment the concentration of DMSO may be at least 5 percent of the substances that are not RNA substances. In another embodiment the concentration of DMSO may be at least 10 percent of the substances that are not RNA substances. In another embodiment the concentration of DMSO may be at least 15 percent of the substances that are not RNA substances. In another embodiment the concentration of DMSO may be at least 20 percent of the substances that are not RNA substances. In another embodiment the concentration of DMSO may be at least 25 percent of the substances that are not RNA substances. In another embodiment the concentration of DMSO may be at least 30 percent of the substances that are not RNA substances. In another embodiment the concentration of DMSO may be at least 35 percent of the substances that are not RNA substances. In another embodiment the concentration of DMSO may be at least 40 percent of the substances that are not RNA substances. In another embodiment the concentration of DMSO may be at least 45 percent of the substances that are not RNA substances. In another embodiment the concentration of DMSO may be at least 50 percent of the substances that are not RNA substances. In another embodiment the concentration of DMSO may be at least 55 percent of the substances that are not RNA substances. In another embodiment the concentration of DMSO may be at least 60 percent of the substances that are not RNA substances. In another embodiment the concentration of DMSO may be at least 65 percent of the substances that are not RNA substances. In another embodiment the concentration of DMSO may be at least 70 percent of the substances that are not RNA substances. In another embodiment the concentration of DMSO may be at least 75 percent of the substances that are not RNA substances. In another embodiment the concentration of DMSO may be at least 80 percent of the substances that are not RNA substances. In another embodiment the concentration of DMSO may be at least 85 percent of the substances that are not RNA substances. In another embodiment the concentration of DMSO may be at least 90 percent of the substances that are not RNA substances. In another embodiment the concentration of DMSO may be at least 95 percent of the substances that are not RNA substances. In another embodiment the concentration of DMSO may be at least 99 percent or even substantially all of the substances that are not RNA substances.

A non-limiting example of an aprotic substance is dimethyl sulfone (also known as DMSO₂, methylsulfonylmethane, MSM, or methyl sulfone). One embodiment of the present invention may include combinations of substances comprising dimethyl sulfone and another embodiment may also comprise at least one RNA substance.

Embodiments of compositions comprising RNA stabilizing substances, of which dimethyl sulfone is a non-limiting example, may have different molar concentrations. For conciseness, the following list of molar concentrations with dimethyl sulfone as an example aprotic substance is herein referred to as the RNA stabilizing substance molar concentration list. In one embodiment the concentration of dimethyl sulfone may be between about 1 nM-10M. In one embodiment the concentration of dimethyl sulfone may be between about 1 nM-9.5M. In one embodiment the concentration of dimethyl sulfone may be between about 1 nM-9M. In one embodiment the concentration of dimethyl sulfone may be between about 1 nM-8.5M. In one embodiment the concentration of dimethyl sulfone may be between about 1 nM-8M. In one embodiment the concentration of dimethyl sulfone may be between about 1 nM-7.5M. In one embodiment the concentration of dimethyl sulfone may be between about 1 nM-7M. In one embodiment the concentration of dimethyl sulfone may be between about 1 nM-6.5M. In one embodiment the concentration of dimethyl sulfone may be between about 1 nM-6M. In one embodiment the concentration of dimethyl sulfone may be between about 1 nM-5.5M. In one embodiment the concentration of dimethyl sulfone may be between about 1 nM-5M. In another embodiment the concentration of dimethyl sulfone may be between about 1 nM-4.5M. In another embodiment the concentration of dimethyl sulfone may be between about 1 nM-4M. In another embodiment the concentration of dimethyl sulfone may be between about 1 nM-3.5M. In another embodiment the concentration of dimethyl sulfone may be between about 1 nM-3M. In another embodiment the concentration of dimethyl sulfone may be between about 1 nM-2.5M. In another embodiment the concentration of dimethyl sulfone may be between about 1 nM-2M. In another embodiment the concentration of dimethyl sulfone may be between about 1 nM-1.5M. In another embodiment the concentration of dimethyl sulfone may be between about 1 nM-1M. In another embodiment the concentration of dimethyl sulfone may be between about 1 nM-500 mM. In another embodiment the concentration of dimethyl sulfone may be between about 1 nM-250 mM. In another embodiment the concentration of dimethyl sulfone may be between about 1 nM-100 mM. In another embodiment the concentration of dimethyl sulfone may be between about 1 nM-50 mM. In another embodiment the concentration of dimethyl sulfone may be between about 1 nM-25 mM. In another embodiment the concentration of dimethyl sulfone may be between about 1 nM-10 mM. In another embodiment the concentration of dimethyl sulfone may be between about 1 nM-5 mM. In another embodiment the concentration of dimethyl sulfone may be between about 1 nM-1 mM. In another embodiment the concentration of dimethyl sulfone may be between about 1 nM-500 μM. In another embodiment the concentration of dimethyl sulfone may be between about 1 nM-250 μM. In another embodiment the concentration of dimethyl sulfone may be between about 1 nM 100 μM. In another embodiment the concentration of dimethyl sulfone may be between about 1 nM-50 μM. In another embodiment the concentration of dimethyl sulfone may be between about 1 nM-25 μM. In another embodiment the concentration of dimethyl sulfone may be between about 1 nM-10 μM. In another embodiment the concentration of dimethyl sulfone may be between about 1 nM-5 μM. In another embodiment the concentration of dimethyl sulfone may be between about 1 nM-1 μM.

In one embodiment the concentration of dimethyl sulfone may be between about 1 μM-10M. In one embodiment the concentration of dimethyl sulfone may be between about 1 μM-9.5M. In one embodiment the concentration of dimethyl sulfone may be between about 1 μM-9M. In one embodiment the concentration of dimethyl sulfone may be between about 1 μM-8.5M. In one embodiment the concentration of dimethyl sulfone may be between about 1 μM-8M. In one embodiment the concentration of dimethyl sulfone may be between about 1 μM-7.5M. In one embodiment the concentration of dimethyl sulfone may be between about 1 μM-7M. In one embodiment the concentration of dimethyl sulfone may be between about 1 μM-6.5M. In one embodiment the concentration of dimethyl sulfone may be between about 1 μM-6M. In one embodiment the concentration of dimethyl sulfone may be between about 1 μM-5.5M. In one embodiment the concentration of dimethyl sulfone may be between about 1 μM-5M. In another embodiment the concentration of dimethyl sulfone may be between about 1 μM-4.5M. In another embodiment the concentration of dimethyl sulfone may be between about 1 μM-4M. In another embodiment the concentration of dimethyl sulfone may be between about 1 μM-3.5M. In another embodiment the concentration of dimethyl sulfone may be between about 1 μM-3M. In another embodiment the concentration of dimethyl sulfone may be between about 1 μM-2.5M. In another embodiment the concentration of dimethyl sulfone may be between about 1 μM-2M. In another embodiment the concentration of dimethyl sulfone may be between about 1 μM-1.5M. In another embodiment the concentration of dimethyl sulfone may be between about 1 μM-1M. In another embodiment the concentration of dimethyl sulfone may be between about 1 μM-500 mM. In another embodiment the concentration of dimethyl sulfone may be between about 1 μM-250 mM. In another embodiment the concentration of dimethyl sulfone may be between about 1 μM-100 mM. In another embodiment the concentration of dimethyl sulfone may be between about 1 μM-50 mM. In another embodiment the concentration of dimethyl sulfone may be between about 1 μM-25 mM. In another embodiment the concentration of dimethyl sulfone may be between about 1 μM-10 mM. In another embodiment the concentration of dimethyl sulfone may be between about 1 μM-5 mM. In another embodiment the concentration of dimethyl sulfone may be between about 1 μM-1 mM. In another embodiment the concentration of dimethyl sulfone may be between about 1 μM-500 μM. In another embodiment the concentration of dimethyl sulfone may be between about 1 μM-250 μM. In another embodiment the concentration of dimethyl sulfone may be between about 1 μM-100 μM. In another embodiment the concentration of dimethyl sulfone may be between about 1 μM-50 μM. In another embodiment the concentration of dimethyl sulfone may be between about 1 μM-25 μM. In another embodiment the concentration of dimethyl sulfone may be between about 1 μM-10 μM. In another embodiment the concentration of dimethyl sulfone may be between about 1 μM-5 μM.

In one embodiment the concentration of dimethyl sulfone may be between about 1 mM-10M. In one embodiment the concentration of dimethyl sulfone may be between about 1 mM-9.5M. In one embodiment the concentration of dimethyl sulfone may be between about 1 mM-9M. In one embodiment the concentration of dimethyl sulfone may be between about 1 mM-8.5M. In one embodiment the concentration of dimethyl sulfone may be between about 1 mM-8M. In one embodiment the concentration of dimethyl sulfone may be between about 1 mM-7.5M. In one embodiment the concentration of dimethyl sulfone may be between about 1 mM-7M. In one embodiment the concentration of dimethyl sulfone may be between about 1 mM-6.5M. In one embodiment the concentration of dimethyl sulfone may be between about 1 mM-6M. In one embodiment the concentration of dimethyl sulfone may be between about 1 mM-5.5M. In another embodiment the concentration of dimethyl sulfone may be between about 1 mM-5M. In another embodiment the concentration of dimethyl sulfone may be between about 1 mM-4.5M. In another embodiment the concentration of dimethyl sulfone may be between about 1 mM-4M. In another embodiment the concentration of dimethyl sulfone may be between about 1 mM-3.5M. In another embodiment the concentration of dimethyl sulfone may be between about 1 mM-3M. In another embodiment the concentration of dimethyl sulfone may be between about 1 mM-2.5M. In another embodiment the concentration of dimethyl sulfone may be between about 1 mM-2M. In another embodiment the concentration of dimethyl sulfone may be between about 1 mM-1.5M. In another embodiment the concentration of dimethyl sulfone may be between about 1 mM-1M. In another embodiment the concentration of dimethyl sulfone may be between about 1 mM-500 mM. In another embodiment the concentration of dimethyl sulfone may be between about 1 mM-250 mM. In another embodiment the concentration of dimethyl sulfone may be between about 1 mM-100 mM. In another embodiment the concentration of dimethyl sulfone may be between about 1 mM-50 mM. In another embodiment the concentration of dimethyl sulfone may be between about 1 mM-25 mM. In another embodiment the concentration of dimethyl sulfone may be between about 1 mM-10 mM. In another embodiment the concentration of dimethyl sulfone may be between about 1 mM-5 mM.

In one embodiment the concentration of dimethyl sulfone may be between about 10 mM-10M. In one embodiment the concentration of dimethyl sulfone may be between about 10 mM-9.5M. In one embodiment the concentration of dimethyl sulfone may be between about 10 mM-9M. In one embodiment the concentration of dimethyl sulfone may be between about 10 mM-8.5M. In one embodiment the concentration of dimethyl sulfone may be between about 10 mM-8M. In one embodiment the concentration of dimethyl sulfone may be between about 10 mM-7.5M. In one embodiment the concentration of dimethyl sulfone may be between about 10 mM-7M. In one embodiment the concentration of dimethyl sulfone may be between about 10 mM-6.5M. In one embodiment the concentration of dimethyl sulfone may be between about 10 mM-6M. In one embodiment the concentration of dimethyl sulfone may be between about 10 mM-5.5M. In another embodiment the concentration of dimethyl sulfone may be between about 10 mM-5M. In another embodiment the concentration of dimethyl sulfone may be between about 10 mM-4.5M. In another embodiment the concentration of dimethyl sulfone may be between about 10 mM-4M. In another embodiment the concentration of dimethyl sulfone may be between about 10 mM-3.5M. In another embodiment the concentration of dimethyl sulfone may be between about 10 mM-3M. In another embodiment the concentration of dimethyl sulfone may be between about 10 mM-2.5M. In another embodiment the concentration of dimethyl sulfone may be between about 10 mM-2M. In another embodiment the concentration of dimethyl sulfone may be between about 10 mM-1.5M. In another embodiment the concentration of dimethyl sulfone may be between about 10 mM-1M. In another embodiment the concentration of dimethyl sulfone may be between about 10 mM-500 mM. In another embodiment the concentration of dimethyl sulfone may be between about 10 mM-250 mM. In another embodiment the concentration of dimethyl sulfone may be between about 10 mM-100 mM. In another embodiment the concentration of dimethyl sulfone may be between about 10 mM-50 mM. In another embodiment the concentration of dimethyl sulfone may be between about 10 mM-25 mM.

In one embodiment the concentration of dimethyl sulfone may be between about 25 mM-10M. In one embodiment the concentration of dimethyl sulfone may be between about 25 mM-9.5M. In one embodiment the concentration of dimethyl sulfone may be between about 25 mM-9M. In one embodiment the concentration of dimethyl sulfone may be between about 25 mM-8.5M. In one embodiment the concentration of dimethyl sulfone may be between about 25 mM-8M. In one embodiment the concentration of dimethyl sulfone may be between about 25 mM-7.5M. In one embodiment the concentration of dimethyl sulfone may be between about 25 mM-7M. In one embodiment the concentration of dimethyl sulfone may be between about 25 mM-6.5M. In one embodiment the concentration of dimethyl sulfone may be between about 25 mM-6M. In one embodiment the concentration of dimethyl sulfone may be between about 25 mM-5.5M. In another embodiment the concentration of dimethyl sulfone may be between about 25 mM-5M. In another embodiment the concentration of dimethyl sulfone may be between about 25 mM-4.5M. In another embodiment the concentration of dimethyl sulfone may be between about 25 mM-4M. In another embodiment the concentration of dimethyl sulfone may be between about 25 mM-3.5M. In another embodiment the concentration of dimethyl sulfone may be between about 25 mM-3M. In another embodiment the concentration of dimethyl sulfone may be between about 25 mM-2.5M. In another embodiment the concentration of dimethyl sulfone may be between about 25 mM-2M. In another embodiment the concentration of dimethyl sulfone may be between about 25 mM-1.5M. In another embodiment the concentration of dimethyl sulfone may be between about 25 mM-1M. In another embodiment the concentration of dimethyl sulfone may be between about 25 mM-500 mM. In another embodiment the concentration of dimethyl sulfone may be between about 25 mM-250 mM. In another embodiment the concentration of dimethyl sulfone may be between about 25 mM-100 mM. In another embodiment the concentration of dimethyl sulfone may be between about 25 mM-50 mM.

In one embodiment the concentration of dimethyl sulfone may be between about 50 mM-10M. In one embodiment the concentration of dimethyl sulfone may be between about 50 mM-9.5M. In one embodiment the concentration of dimethyl sulfone may be between about 50 mM-9M. In one embodiment the concentration of dimethyl sulfone may be between about 50 mM-8.5M. In one embodiment the concentration of dimethyl sulfone may be between about 50 mM-8M. In one embodiment the concentration of dimethyl sulfone may be between about 50 mM-7.5M. In one embodiment the concentration of dimethyl sulfone may be between about 50 mM-7M. In one embodiment the concentration of dimethyl sulfone may be between about 50 mM-6.5M. In one embodiment the concentration of dimethyl sulfone may be between about 50 mM-6M. In one embodiment the concentration of dimethyl sulfone may be between about 50 mM-5.5M. In another embodiment the concentration of dimethyl sulfone may be between about 50 mM-5M. In another embodiment the concentration of dimethyl sulfone may be between about 50 mM-4.5M. In another embodiment the concentration of dimethyl sulfone may be between about 50 mM-4M. In another embodiment the concentration of dimethyl sulfone may be between about 50 mM-3.5M. In another embodiment the concentration of dimethyl sulfone may be between about 50 mM-3M. In another embodiment the concentration of dimethyl sulfone may be between about 50 mM-2.5M. In another embodiment the concentration of dimethyl sulfone may be between about 50 mM-2M. In another embodiment the concentration of dimethyl sulfone may be between about 50 mM-1.5M. In another embodiment the concentration of dimethyl sulfone may be between about 50 mM-1M. In another embodiment the concentration of dimethyl sulfone may be between about 50 mM-500 mM. In another embodiment the concentration of dimethyl sulfone may be between about 50 mM-250 mM. In another embodiment the concentration of dimethyl sulfone may be between about 50 mM-100 mM.

In one embodiment the concentration of dimethyl sulfone may be between about 100 mM-10M. In one embodiment the concentration of dimethyl sulfone may be between about 100 mM-9.5M. In one embodiment the concentration of dimethyl sulfone may be between about 100 mM-9M. In one embodiment the concentration of dimethyl sulfone may be between about 100 mM-8.5M. In one embodiment the concentration of dimethyl sulfone may be between about 100 mM-8M. In one embodiment the concentration of dimethyl sulfone may be between about 100 mM-7.5M. In one embodiment the concentration of dimethyl sulfone may be between about 100 mM-7M. In one embodiment the concentration of dimethyl sulfone may be between about 100 mM-6.5M. In one embodiment the concentration of dimethyl sulfone may be between about 100 mM-6M. In one embodiment the concentration of dimethyl sulfone may be between about 100 mM-5.5M. In another embodiment the concentration of dimethyl sulfone may be between about 100 mM-5M. In another embodiment the concentration of dimethyl sulfone may be between about 100 mM-4.5M. In another embodiment the concentration of dimethyl sulfone may be between about 100 mM-4M. In another embodiment the concentration of dimethyl sulfone may be between about 100 mM-3.5M. In another embodiment the concentration of dimethyl sulfone may be between about 100 mM-3M. In another embodiment the concentration of dimethyl sulfone may be between about 100 mM-2.5M. In another embodiment the concentration of dimethyl sulfone may be between about 100 mM-2M. In another embodiment the concentration of dimethyl sulfone may be between about 100 mM-1.5M. In another embodiment the concentration of dimethyl sulfone may be between about 100 mM-1M. In another embodiment the concentration of dimethyl sulfone may be between about 100 mM-500 mM. In another embodiment the concentration of dimethyl sulfone may be between about 100 mM-250 mM.

In one embodiment the concentration of dimethyl sulfone may be between about 250 mM-10M. In one embodiment the concentration of dimethyl sulfone may be between about 250 mM-9.5M. In one embodiment the concentration of dimethyl sulfone may be between about 250 mM-9M. In one embodiment the concentration of dimethyl sulfone may be between about 250 mM-8.5M. In one embodiment the concentration of dimethyl sulfone may be between about 250 mM-8M. In one embodiment the concentration of dimethyl sulfone may be between about 250 mM-7.5M. In one embodiment the concentration of dimethyl sulfone may be between about 250 mM-7M. In one embodiment the concentration of dimethyl sulfone may be between about 250 mM-6.5M. In one embodiment the concentration of dimethyl sulfone may be between about 250 mM-6M. In one embodiment the concentration of dimethyl sulfone may be between about 250 mM-5.5M. In another embodiment the concentration of dimethyl sulfone may be between about 250 mM-5M. In another embodiment the concentration of dimethyl sulfone may be between about 250 mM-4.5M. In another embodiment the concentration of dimethyl sulfone may be between about 250 mM-4M. In another embodiment the concentration of dimethyl sulfone may be between about 250 mM-3.5M. In another embodiment the concentration of dimethyl sulfone may be between about 250 mM-3M. In another embodiment the concentration of dimethyl sulfone may be between about 250 mM-2.5M. In another embodiment the concentration of dimethyl sulfone may be between about 250 mM-2M. In another embodiment the concentration of dimethyl sulfone may be between about 250 mM-1.5M. In another embodiment the concentration of dimethyl sulfone may be between about 250 mM-1M. In another embodiment the concentration of dimethyl sulfone may be between about 250 mM-500 mM.

In one embodiment the concentration of dimethyl sulfone may be between about 500 mM-10M. In one embodiment the concentration of dimethyl sulfone may be between about 500 mM-9.5M. In one embodiment the concentration of dimethyl sulfone may be between about 500 mM-9M. In one embodiment the concentration of dimethyl sulfone may be between about 500 mM-8.5M. In one embodiment the concentration of dimethyl sulfone may be between about 500 mM-8M. In one embodiment the concentration of dimethyl sulfone may be between about 500 mM-7.5M. In one embodiment the concentration of dimethyl sulfone may be between about 500 mM-7M. In one embodiment the concentration of dimethyl sulfone may be between about 500 mM-6.5M. In one embodiment the concentration of dimethyl sulfone may be between about 500 mM-6M. In one embodiment the concentration of dimethyl sulfone may be between about 500 mM-5.5M. In another embodiment the concentration of dimethyl sulfone may be between about 500 mM-5M. In another embodiment the concentration of dimethyl sulfone may be between about 500 mM-4.5M. In another embodiment the concentration of dimethyl sulfone may be between about 500 mM-4M. In another embodiment the concentration of dimethyl sulfone may be between about 500 mM-3.5M. In another embodiment the concentration of dimethyl sulfone may be between about 500 mM-3M. In another embodiment the concentration of dimethyl sulfone may be between about 500 mM-2.5M. In another embodiment the concentration of dimethyl sulfone may be between about 500 mM-2M. In another embodiment the concentration of dimethyl sulfone may be between about 500 mM-1.5M. In another embodiment the concentration of dimethyl sulfone may be between about 500 mM-1M.

In one embodiment the concentration of dimethyl sulfone may be between about 1M-10M. In one embodiment the concentration of dimethyl sulfone may be between about 1M-9.5M. In one embodiment the concentration of dimethyl sulfone may be between about 1M-9M. In one embodiment the concentration of dimethyl sulfone may be between about 1M-8.5M. In one embodiment the concentration of dimethyl sulfone may be between about 1M-8M. In one embodiment the concentration of dimethyl sulfone may be between about 1M-7.5M. In one embodiment the concentration of dimethyl sulfone may be between about 1M-7M. In one embodiment the concentration of dimethyl sulfone may be between about 1M-6.5M. In one embodiment the concentration of dimethyl sulfone may be between about 1M-6M. In one embodiment the concentration of dimethyl sulfone may be between about 1M-5.5M. In another embodiment the concentration of dimethyl sulfone may be between about 1M-5M. In another embodiment the concentration of dimethyl sulfone may be between about 1M-4.5M. In another embodiment the concentration of dimethyl sulfone may be between about 1M-4M. In another embodiment the concentration of dimethyl sulfone may be between about 1M-3.5M. In another embodiment the concentration of dimethyl sulfone may be between about 1M-3M. In another embodiment the concentration of dimethyl sulfone may be between about 1M-2.5M. In another embodiment the concentration of dimethyl sulfone may be between about 1M-2M.

In one embodiment a combination comprising at least one aprotic substance and at least one RNA substance has a melting point of about 19° C. or less.

In another embodiment, a combination comprising at least one aprotic substance and at least one RNA substance is stored or used below the melting point of the combination of substances.

In one embodiment a combination comprising at least one aprotic substance and at least one RNA substance is a material that has a melting point at atmospheric pressure that may be between about −80° C. and about 200° C.

In one embodiment a combination comprising at least one aprotic substance and at least one RNA substance is a material that has a melting point at atmospheric pressure that may be between about −50° C. and about 150° C.

In one embodiment a combination comprising least one aprotic substance and at least one RNA substance is a material that has a melting point at atmospheric pressure that may be between about −25° C. and about 120° C.

In one embodiment a combination comprising at least one aprotic substance and at least one RNA substance is a material that has a melting point at atmospheric pressure that may be between about −25° C. and about 50° C.

In one embodiment a combination comprising at least one aprotic substance and at least one RNA substance is a material that has a melting point at atmospheric pressure that may be between about 0° C. and about 50° C.

In one embodiment a combination comprising at least one aprotic substance and at least one RNA substance is a material that has a melting point at atmospheric pressure that may be between about 0° C. and about 40° C.

In one embodiment a combination comprising at least one aprotic substance and at least one RNA substance is a material that has a melting point at atmospheric pressure that may be between about 0° C. and about 30° C. In one embodiment a combination comprising at least one aprotic substance and at least one RNA substance is a material that has a melting point at atmospheric pressure that may be between about 10° C. and about 30° C.

In one embodiment a combination comprising at least one aprotic substance and at least one RNA substance is a material that has a melting point at atmospheric pressure that may be between about 15° C. and about 25° C. In one embodiment that has a melting point between about 15° C. and about 25° C. at least one aprotic substance in the combination is DMSO.

In one embodiment a combination comprising at least one aprotic substance and at least one RNA substance is a material that has a melting point at atmospheric pressure that may be between about 10° C. and about 25° C.

In one embodiment a combination comprising at least diethyl sulfoxide and at least one RNA substance, the combination has a melting point between about 10° C. and about 25° C.

A non-limiting example of an aprotic substance is diethyl sulfoxide (DESO), wherein diethyl sulfoxide may be substituted for or used in combination with DMSO or dimethyl sulfone as described above. One embodiment of the present invention may include combinations of substances comprising diethyl sulfoxide and another embodiment may also comprise at least one RNA substance. Other embodiments may include combinations of substances comprising diethyl sulfoxide wherein the concentration of diethyl sulfoxide may be a concentration in the RNA stabilizing substance weight percent concentration list or in the RNA stabilizing substance molar concentration list. Other embodiments may include combinations of substances comprising diethyl sulfoxide, DMSO, or dimethyl sulfone.

In one embodiment a combination comprising at least diethyl sulfoxide the combination has a melting point between about 10° C. and about 25° C.

A non-limiting example of an aprotic substance that may be used is dimethyl sulfoxide (DMSO), substituted for or used in combination with dimethyl sulfone as described above. One embodiment of the present invention may include combinations of substances comprising DMSO and another embodiment may also comprise at least one RNA substance. Other embodiments may include combinations of substances comprising DMSO wherein the concentration of DMSO may be a concentration in the RNA stabilizing substance weight percent concentration list or in the RNA stabilizing substance molar concentration list.

A non-limiting example of an aprotic substance that may be used is dimethyl sulfone (also known as DMSO₂, methylsulfonylmethane, MSM, or methyl sulfone), substituted for or used in combination with DMSO as described above. One embodiment of the present invention may include combinations of substances comprising dimethyl sulfone and another embodiment may also comprise at least one RNA substance. Other embodiments may include combinations of substances comprising dimethyl sulfone wherein the concentration of dimethyl sulfone may be a concentration in the RNA stabilizing substance weight percent concentration list or in the RNA stabilizing substance molar concentration list.

A non-limiting example of an aprotic substance that may be used is N-Methyl-2-pyrrolidone (also known as, 1-Methyl-2-pyrrolidinone, or NMP), substituted for or used in combination with DMSO or dimethyl sulfone as described above. One embodiment of the present invention may include combinations of substances comprising NMP and another embodiment may also comprise at least one RNA substance. Other embodiments may include combinations of substances comprising NMP wherein the concentration of NMP may be a concentration in the RNA stabilizing substance weight percent concentration list or in the RNA stabilizing substance molar concentration list.

In one embodiment a combination comprising at least one RNA stabilizing substance, such as an aprotic substance, and at least one RNA substance has a melting point of about 19° C. or less.

In another embodiment, a combination comprising at least one RNA stabilizing substance, such as an aprotic substance, and at least one RNA substance is stored or used below the melting point of the combination of substances.

In one embodiment a combination comprising at least one RNA stabilizing substance, such as an aprotic substance, and at least one RNA substance is a material that has a melting point at atmospheric pressure that may be between about −80° C. and about 200° C.

In one embodiment a combination comprising at least one RNA stabilizing substance, such as an aprotic substance, and at least one RNA substance is a material that has a melting point at atmospheric pressure that may be between about −50° C. and about 150° C.

In one embodiment a combination comprising at least one RNA stabilizing substance, such as an aprotic substance, and at least one RNA substance is a material that has a melting point at atmospheric pressure that may be between about −25° C. and about 120° C.

In one embodiment a combination comprising at least one RNA stabilizing substance, such as an aprotic substance, and at least one RNA substance is a material that has a melting point at atmospheric pressure that may be between about −25° C. and about 50° C.

In one embodiment a combination comprising at least one RNA stabilizing substance, such as an aprotic substance, and at least one RNA substance is a material that has a melting point at atmospheric pressure that may be between about 0° C. and about 50° C.

In one embodiment a combination comprising at least one RNA stabilizing substance, such as an aprotic substance, and at least one RNA substance is a material that has a melting point at atmospheric pressure that may be between about 0° C. and about 40° C.

In one embodiment a combination comprising at least one RNA stabilizing substance, such as an aprotic substance, and at least one RNA substance is a material that has a melting point at atmospheric pressure that may be between about 0° C. and about 30° C. In one embodiment a combination comprising at least one RNA stabilizing substance, such as an aprotic substance, and at least one RNA substance is a material that has a melting point at atmospheric pressure that may be between about 10° C. and about 30° C.

In one embodiment a combination comprising at least one RNA stabilizing substance, such as an aprotic substance, and at least one RNA substance is a material that has a melting point at atmospheric pressure that may be between about 15° C. and about 25° C.

In one embodiment a combination comprising at least one RNA substance and DMSO has a melting point between about 15° C. and about 25° C.

In one embodiment a combination comprising at least one RNA stabilizing substance, such as an aprotic substance, and at least one RNA substance is a material that has a melting point at atmospheric pressure that may be between about 10° C. and about 25° C.

In one embodiment a combination comprising at least one RNA substance and diethyl sulfoxide has a melting point between about 10° C. and about 25° C.

In one embodiment one or more aprotic substance may comprise one or more ester. In one embodiment one or more aprotic substance may comprise one or more carboxylate ester. In one embodiment one or more aprotic substance may comprise one or more acyclic carboxylate ester. In one embodiment one or more aprotic substance may comprise one or more cyclic carboxylate ester.

In one embodiment one more aprotic substance may comprise a carboxylate ester comprising the formula Z₁O(C═O)Z₂, wherein Z₁ and Z₂ are independently selected Z groups comprising at least one carbon atom, and Z₁ or Z₂ may be the same or different.

In a non-limiting example of an aprotic carboxylate ester, Z₁ may be a butyl group comprising four carbon atoms and Z₂ may be a methyl group comprising one carbon, wherein an aprotic carboxylate ester may be butyl acetate.

In one embodiment one or more aprotic substance may comprise a carbonate ester. In one embodiment one or more aprotic substance may comprise one or more acyclic carbonate ester. In one embodiment one or more aprotic substance may comprise one or more cyclic carbonate ester.

In one embodiment one more aprotic substance may comprise a carbonate ester comprising the formula Z₁O(C═O)OZ₂, wherein Z₁ and Z₂ are independently selected Z groups comprising at least one carbon atom, and Z₁ or Z₂ may be the same or different.

In a non-limiting example of an aprotic carbonate ester, Z₁ may be an ethyl group comprising two carbon atoms and Z₂ may be an ethyl group comprising two carbons, wherein an aprotic carbonate ester may be diethyl carbonate.

In one embodiment a Z group may be comprised of at least one carbon atom, wherein Z may be comprised of C1-16 alkyl, alkenyl, alkynyl, aryl, or aralkyl group, each of which may contain up to 8 heteroatoms. In another embodiment a Z group may be comprised of C1-12 alkyl, alkenyl, alkynyl, aryl, or aralkyl group, each of which may contain up to 6 heteroatoms. In another embodiment a Z group may be comprised of C1-8 alkyl, alkenyl, alkynyl, aryl, or aralkyl group, each of which may contain up to 4 heteroatoms. In another embodiment a Z group may be comprised of C1-6 alkyl, alkenyl, alkynyl, aryl, or aralkyl group, each of which may contain up to 3 heteroatoms.

In another embodiment one or more Z groups may be aprotic. In another embodiment one or more Z groups may form a ring structure. In another embodiment one or more Z groups may form at least part of a ring structure. In another embodiment one or more Z groups may be at least part of a ring structure. In another embodiment one or more Z groups may comprise at least part of a ring structure.

In one embodiment a Z group may be covalently bonded to another Z group. In one embodiment one or more Z group may form a heterocyclic ring structure. In one embodiment one or more Z group may form at least part of a heterocyclic ring structure. In one embodiment one or more Z group may be at least part of a heterocyclic ring structure. In one embodiment one or more Z group may comprise at least part of a heterocyclic ring structure.

A non-limiting example of an aprotic substance comprised of a carboxylate ester that may be used is butyl acetate (also known as butyl ethanoate), wherein butyl acetate may be substituted for or used in combination with DMSO or dimethyl sulfone as described above. One embodiment of the present invention may include combinations of substances comprising butyl acetate and another embodiment may also comprise at least one RNA substance. Other embodiments may include combinations of substances comprising butyl acetate wherein the concentration of butyl acetate may be a concentration in the RNA stabilizing substance weight percent concentration list or in the RNA stabilizing substance molar concentration list.

A non-limiting example of an aprotic substance comprised of a carbonate ester that may be used is diethyl carbonate (also known as DEC), wherein diethyl carbonate may be substituted for or used in combination with DMSO or dimethyl sulfone as described above. One embodiment of the present invention may include combinations of substances comprising diethyl carbonate and another embodiment may also comprise at least one RNA substance. Other embodiments may include combinations of substances comprising diethyl carbonate wherein the concentration of diethyl carbonate may be a concentration in the RNA stabilizing substance weight percent concentration list or in the RNA stabilizing substance molar concentration list.

A non-limiting example of an aprotic substance comprised of a cyclic carbonate ester that may be used is propylene carbonate (also known as 4-methyl-1,3-dioxolan-2-one or 1,2-propanediol cyclic carbonate), wherein propylene carbonate may be substituted for or used in combination with DMSO or dimethyl sulfone as described above. One embodiment of the present invention may include combinations of substances comprising propylene carbonate and another embodiment may also comprise at least one RNA substance. Other embodiments may include combinations of substances comprising propylene carbonate wherein the concentration of propylene carbonate may be a concentration in the RNA stabilizing substance weight percent concentration list or in the RNA stabilizing substance molar concentration list.

In one embodiment one or more aprotic substance may comprise glycerine acetate.

A non-limiting example of an aprotic substance comprising glycerin acetate that may be used is triacetin (also known as 1,2,3-triacetylglycerol or glycerin triacetate), wherein triacetin may be substituted for or used in combination with DMSO or dimethyl sulfone as described above. One embodiment of the present invention may include combinations of substances comprising triacetin and another embodiment may also comprise at least one RNA substance. Other embodiments may include combinations of substances comprising triacetin wherein the concentration of triacetin may be a concentration in the RNA stabilizing substance weight percent concentration list or in the RNA stabilizing substance molar concentration list.

In one embodiment an aprotic substance may comprise one or more choline-based ester.

A non-limiting example of an aprotic substance comprised of one or more choline-based ester that may be used is acetylcholine, wherein acetylcholine may be substituted for or used in combination with DMSO or dimethyl sulfone as described above. One embodiment of the present invention may include combinations of substances comprising acetylcholine and another embodiment may also comprise at least one RNA substance. Other embodiments may include combinations of substances comprising acetylcholine wherein the concentration of acetylcholine may be a concentration in the RNA stabilizing substance weight percent concentration list or in the RNA stabilizing substance molar concentration list.

A non-limiting example of an aprotic substance comprised of one or more choline-based ester that may be used is butyrylcholine, wherein butyrylcholine may be substituted for or used in combination with DMSO or dimethyl sulfone as described above. One embodiment of the present invention may include combinations of substances comprising butyrylcholine and another embodiment may also comprise at least one RNA substance. Other embodiments may include combinations of substances comprising butyrylcholine wherein the concentration of butyrylcholine may be a concentration in the RNA stabilizing substance weight percent concentration list or in the RNA stabilizing substance molar concentration list.

In one embodiment an aprotic substance may comprise a diester comprising the formula Z₁O(C═O)Cₙ(C═O)OZ₂, wherein Z₁ and Z₂ are independently selected Z groups comprising at least one carbon atom, and Z₁ or Z₂ may be the same or different.

In one embodiment n may be an integer between 0-10, wherein n may be 0, 1, 2, 3, 4, 5, 6, 7, 8, 9, or 10. In one embodiment n may be an integer between 0-5, wherein n may be 0, 1, 2, 3, 4, or 5.

In a non-limiting example of an aprotic diester, Z₁ may be a methyl group comprising one carbon atom, n=1, and Z₂ may be a methyl group comprising one carbon, wherein an aprotic diester may be dimethyl malonate.

In another non-limiting example of an aprotic diester, Z₁ may be a methyl group comprising one carbon atom, n=0, and Z₂ may be a methyl group comprising one carbon, wherein an aprotic diester may be dimethyl oxalate.

A non-limiting example of an aprotic substance comprising a diester that may be used is dimethyl malonate (also known as dimethyl propanedioate or malonic acid dimethyl ester), substituted for or used in combination with DMSO or dimethyl sulfone as described above. One embodiment of the present invention may include combinations of substances comprising dimethyl malonate and another embodiment may also comprise at least one RNA substance. Other embodiments may include combinations of substances comprising dimethyl malonate wherein the concentration of dimethyl malonate may be a concentration in the RNA stabilizing substance weight percent concentration list or in the RNA stabilizing substance molar concentration list.

A non-limiting example of an aprotic substance comprising a diester that may be used is diethyl malonate (also known as diethyl propanedioate or DEM), substituted for or used in combination with DMSO or dimethyl sulfone as described above. One embodiment of the present invention may include combinations of substances comprising diethyl malonate and another embodiment may also comprise at least one RNA substance. Other embodiments may include combinations of substances comprising diethyl malonate wherein the concentration of diethyl malonate may be a concentration in the RNA stabilizing substance weight percent concentration list or in the RNA stabilizing substance molar concentration list.

A non-limiting example of an aprotic substance comprising a diester that may be used is dimethyl oxalate, substituted for or used in combination with DMSO or dimethyl sulfone as described above. One embodiment of the present invention may include combinations of substances comprising dimethyl oxalate and another embodiment may also comprise at least one RNA substance. Other embodiments may include combinations of substances comprising dimethyl oxalate wherein the concentration of dimethyl oxalate may be a concentration in the RNA stabilizing substance weight percent concentration list or in the RNA stabilizing substance molar concentration list.

For conciseness, the following list of aprotic substances is herein referred to as the aprotic substance list wherein one or more of the following substances may be substituted for or used in combination with one or more aprotic substance as described herein.

In other embodiments, other aprotic substances that may be substituted for or used in combination with DMSO or dimethyl sulfone as described above may include, but are not limited to: DMSO, diethyl sulfoxide, dimethyl sulfone, triacetin, N-methyl-2-pyrrolidone, acetylcholine, butyrylcholine, methacholine, diethyl carbonate, propylene carbonate, ethyl acetate, methyl benzoate, ethylene carbonate, dimethyl carbonate, ethylmethyl carbonate, ethyl pentanoate, butyl acetate, isobutyl acetate, isobutyl valerate, methyl butyrate, phenylethyl acetate, propyl butyrate, bornyl acetate, diethyl oxalate, dibutyl oxalate, tetraethyl pentane-1,3,3,5-tetracarboxylate, tetramethyl butane-1,1,4,4-tetracarboxylate, diethyl succinosuccinate, diethyl succinate, ethyl acetoacetate, ethyl benzoate, ethyl benzoylacetate, benzyl acetate, methyl acetate, sulfolane, dimethyl malonate, diethyl malonate, dimethyl oxalate, diethyl oxalate, diphenyl oxalate, methyl dihydrojasmonate, methyl jasmonate, or acetone, or mixtures, or combinations thereof.

Embodiments of the present invention may include combinations comprising one or more substance selected from the aprotic substance list substituted for or used in combination with DMSO or dimethyl sulfone and other embodiments may also comprise at least one RNA substance.

Other embodiments may include combinations of substances comprising one or more aprotic substance selected from the aprotic substance list wherein the concentration of the substances may be a concentration in the RNA stabilizing substance weight percent concentration list or in the RNA stabilizing substance molar concentration list.

Embodiments of the present invention may comprise one or more aprotic substance selected from the preferred aprotic substance list below.

For conciseness, the following list of aprotic substances is herein referred to as the preferred aprotic substance list, wherein one or more of the following substances may be substituted for or used in combination with one or more aprotic substance as described herein, including but not limited to: DMSO, diethyl sulfoxide, dimethyl sulfone, sulfolane, N-methyl-2-pyrrolidone, triacetin, acetylcholine, butyrylcholine, methacholine, diethyl carbonate, dimethyl oxalate, propylene carbonate, ethyl acetate, butyl acetate, benzyl acetate, dimethyl malonate, diethyl malonate, methyl dihydrojasmonate, methyl jasmonate, acetone, or methyl benzoate, or mixtures, or combinations thereof.

Embodiments of the present invention may include combinations comprising one or more substance selected from the preferred aprotic substance list substituted for or used in combination with DMSO or dimethyl sulfone and other embodiments may also comprise at least one RNA substance.

Other embodiments may include combinations of substances comprising one or more aprotic substance selected from the preferred aprotic substance list wherein the concentration of the substances may be a concentration in the RNA stabilizing substance weight percent concentration list or in the RNA stabilizing substance molar concentration list.

In another embodiment one or more aprotic substance, such as DMSO, may be used in a composition comprising a combination of one or more RNA substance, one or more aprotic substance, and water. In another embodiment any composition comprising one or more aprotic substance and one or more RNA substance may also comprise one or more cellular uptake agent, as described herein.

In one embodiment, an aprotic substance may have a molecular weight between about 25-1,000,000 daltons. In one embodiment, an aprotic substance may have a molecular weight between about 25-500,000 daltons. In one embodiment, an aprotic substance may have a molecular weight between about 25-200,000 daltons. In one embodiment, an aprotic substance may have a molecular weight between about 25-100,000 daltons. In one embodiment, an aprotic substance may have a molecular weight between about 25-50,000 daltons. In one embodiment, an aprotic substance may have a molecular weight between about 25-25,000 daltons. In one embodiment, an aprotic substance may have a molecular weight between about 25-10,000 daltons. In one embodiment, an aprotic substance may have a molecular weight between about 25-5,000 daltons. In one embodiment, an aprotic substance may have a molecular weight between about 25-2,000 daltons. In one embodiment, an aprotic substance may have a molecular weight between about 25-1,000 daltons. In one embodiment, an aprotic substance may have a molecular weight between about 25-900 daltons. In one embodiment, an aprotic substance may have a molecular weight between about 25-800 daltons. In one embodiment, an aprotic substance may have a molecular weight between about 25-700 daltons. In one embodiment, an aprotic substance may have a molecular weight between about 25-600 daltons. In one embodiment, an aprotic substance may have a molecular weight between about 25-500 daltons. In one embodiment, an aprotic substance may have a molecular weight between about 25-400 daltons. In one embodiment, an aprotic substance may have a molecular weight between about 25-300 daltons. In one embodiment, an aprotic substance may have a molecular weight between about 25-250 daltons.

In one embodiment, an aprotic substance may have a molecular weight between about 50-1,000,000 daltons. In one embodiment, an aprotic substance may have a molecular weight between about 50-500,000 daltons. In one embodiment, an aprotic substance may have a molecular weight between about 50-200,000 daltons. In one embodiment, an aprotic substance may have a molecular weight between about 50-100,000 daltons. In one embodiment, an aprotic substance may have a molecular weight between about 50-50,000 daltons. In one embodiment, an aprotic substance may have a molecular weight between about 50-25,000 daltons. In one embodiment, an aprotic substance may have a molecular weight between about 50-10,000 daltons. In one embodiment, an aprotic substance may have a molecular weight between about 50-5,000 daltons. In one embodiment, an aprotic substance may have a molecular weight between about 50-2,000 daltons. In one embodiment, an aprotic substance may have a molecular weight between about 50-1,000 daltons. In one embodiment, an aprotic substance may have a molecular weight between about 50-900 daltons. In one embodiment, an aprotic substance may have a molecular weight between about 50-800 daltons. In one embodiment, an aprotic substance may have a molecular weight between about 50-700 daltons. In one embodiment, an aprotic substance may have a molecular weight between about 50-600 daltons. In one embodiment, an aprotic substance may have a molecular weight between about 50-500 daltons. In one embodiment, an aprotic substance may have a molecular weight between about 50-400 daltons. In one embodiment, an aprotic substance may have a molecular weight between about 50-300 daltons. In one embodiment, an aprotic substance may have a molecular weight between about 50-250 daltons.

One embodiment of the present invention is the method whereby one or more aprotic substance may be combined, such as by mixing, with at least one or more RNA substance to produce a mixture comprising at least one or more aprotic substance and at least one or more RNA substance. As a non-limiting example one or more RNA substance may be mixed with one or more aprotic substance to produce a mixture comprising at least one or more aprotic substance and at least one or more RNA substance.

Another embodiment of the present invention is the method whereby one or more aprotic substance may be combined, such as by mixing, with at least one or more RNA substance to produce a mixture comprising at least one or more RNA substance and at least one or more aprotic substance at one or more of the RNA stabilizing substance concentrations within the RNA stabilizing substance molar concentration list or RNA stabilizing substance weight percent concentration list as described herein. These same methods may be used to combine other substances, including, but not limited to one or more cellular uptake agents, with one or more RNA substances and one or more aprotic substances to produce a mixture comprising one or more RNA substance, one or more aprotic substance, and one or more cellular uptake agent.

Another embodiment of the present invention is the method whereby one or more aprotic substance may be combined, such as by mixing, with at least one or more RNA substance to produce a mixture comprising at least one or more RNA substance and at least one or more aprotic substance at one or more of the RNA stabilizing substance concentrations within the RNA stabilizing substance molar concentration list or RNA stabilizing substance weight percent concentration list as described herein. These same methods may also be used to combine one or more RNA substances, one or more RNA stabilizing substances, and one or more additional other substances and may include one or more cellular uptake agents, with one or more chelating agents, one or more buffering agents, one or more salts, water or other substances. Other non-limiting embodiments of the present invention are mixtures comprising one or more RNA substances, one or more RNA stabilizing substances, and one or more additional other substances and may include one or more cellular uptake agents, with one or more chelating agents, one or more buffering agents, one or more salts, water or other substances.

One embodiment of the present invention is the method whereby one or more aprotic substance may be combined, such as by mixing, with at least one or more RNA substance to produce a composition comprising at least one or more RNA substance and at least one or more aprotic substance. Another embodiment of the present invention is the method whereby one or more aprotic substance may be combined, such as by mixing, with one or more RNA substance to produce a composition comprising at least one or more RNA substance and at least one or more aprotic substance at one or more of the RNA stabilizing substance concentrations within the RNA stabilizing substance molar concentration list or RNA stabilizing substance weight percent concentration list as described herein. These same methods may be used to combine other substances, including, but not limited to cellular uptake agents, with one or more RNA substances and one or more aprotic substances to produce a composition comprising one or more RNA substance, one or more aprotic substance, and one or more cellular uptake agent.

The inventors have discovered that mixtures comprising at least one or more RNA substance and at least one or more RNA stabilizing substance improves RNA stability. The inventors have discovered that RNA stability may be improved with mixtures comprising at least one or more RNA substance and multiple RNA stabilizing substances in which the number of multiple RNA substances is preferably between two and five. As a non-limiting example, RNA stability may be improved with mixtures comprising at least one or more RNA substance and multiple RNA stabilizing substances where the number of multiple RNA stabilizing substances is five.

Embodiments of the present invention may be compositions that comprise at least one RNA stabilizing substance, at least one RNA substance, and water. These embodiments comprising water may be any composition as described herein that may also comprise water.

In one embodiment an RNA stabilizing substance may comprise at least one sulfur atom. In one embodiment an aprotic substance may comprise at least one sulfur atom.

In one embodiment an RNA stabilizing substance may comprise a polar aprotic substance. In one embodiment an aprotic substance may comprise a polar aprotic substance. In one embodiment a polar aprotic substance may comprise at least one oxygen atom. In one embodiment a polar aprotic substance may comprise at least one nitrogen atom. In one embodiment a polar aprotic substance may comprise at least one sulfur atom.

Embodiments of the present invention may be compositions that comprise at least one RNA stabilizing substance, at least one RNA substance, and one or more buffering agent. These embodiments comprising one or more buffering agent may be any composition as described herein that may also comprise one or more buffering agent.

Non-limiting examples of one or more buffering agent that may be used, may comprise but are not limited to, one or more of the following: phosphate, citric acid, citrate, acetic acid, acetate, tris, bis-tris, carbonate, bicarbonate, imidazole, MES, ADA, ACES, PIPES, MOPSO, bis-tris propane, BES, MOPS, TES, HEPES, DIPSO, MOBS, TAPSO, HEPPSO, POPSO, TEA, EPPS, tricine, glycine, diglycine, bicine, HEPBS, TAPS, AMPD, TABS, AMPSO, CHES, CAPSO, AMP, CAPS, or CABS, or mixtures, or combinations thereof.

In one embodiment a buffering agent may be present in concentrations from about 1 mM to 5M, such as about 1 mM, 5 mM, 10 mM, 20 mM, 30 mM, 40 mM, 50 mM, 100 mM, 200 mM, 300 mM, 400 mM, 500 mM, 750 mM, 1M, 1.5M, 2M, 2.5M, 3M, 3.5M, 4M, 4.5M, or 5M. In one embodiment a buffering agent may be present in concentrations from about 1 mM to 1M, such as about 1 mM, 5 mM, 10 mM, 20 mM, 30 mM, 40 mM, 50 mM, 100 mM, 200 mM, 300 mM, 400 mM, 500 mM, 750 mM, or 1M. In another embodiment a buffering agent may be present in concentrations from about 10 mM-1M, such as about 10 mM, 20 mM, 30 mM, 40 mM, 50 mM, 100 mM, 200 mM, 300 mM, 400 mM, 500 mM, 750 mM, or 1M. In another embodiment a buffering agent may be present from about 10 mM-500 mM, such as about 10 mM, 20 mM, 30 mM, 40 mM, 50 mM, 100 mM, 200 mM, 300 mM, 400 mM, or 500 mM. In another embodiment a buffering agent may be present from about 10 mM-200 mM, such as about 10 mM, 20 mM, 30 mM, 40 mM, 50 mM, 100 mM, or 200 mM. In another embodiment a buffering agent may be present from about 10 mM-100 mM, such as about 10 mM, 20 mM, 30 mM, 40 mM, 50 mM, or 100 mM.

In one embodiment a buffering agent may have a molecular weight between about 50 daltons-100,000 daltons. In one embodiment a buffering agent may have a molecular weight between about 50 daltons-50,000 daltons. In one embodiment a buffering agent may have a molecular weight between about 50 daltons-25,000 daltons. In one embodiment a buffering agent may have a molecular weight between about 50 daltons-10,000 daltons. In one embodiment a buffering agent may have a molecular weight between about 50 daltons-5,000 daltons. In one embodiment a buffering agent may have a molecular weight between about 50 daltons-1,000 daltons. In one embodiment a buffering agent may have a molecular weight between about 50 daltons-750 daltons. In one embodiment a buffering agent may have a molecular weight between about 50 daltons-500 daltons. In one embodiment a buffering agent may have a molecular weight between about 50 daltons-350 daltons. In one embodiment a buffering agent may have a molecular weight between about 100 daltons-750 daltons. In one embodiment a buffering agent may have a molecular weight between about 100 daltons-500 daltons. In one embodiment a buffering agent may have a molecular weight between about 100 daltons-350 daltons.

Embodiments of the present invention may be compositions that comprise at least one RNA stabilizing substance, at least one RNA substance, and one or more salt. These embodiments comprising one or more salt may be any composition as described herein that may also comprise one or more salt. In one embodiment the one or more salt may comprise an inorganic salt.

In one embodiment, the one or more salt may comprise at least one monovalent cation or at least one divalent cation. In one embodiment, the one or more salt may comprise at least one monovalent anion or at least one divalent anion.

Non-limiting examples of the one or more salt that may be used, may comprise but are not limited to, one or more of the following: sodium chloride, potassium chloride, magnesium chloride, or calcium chloride, or mixtures, or combinations thereof.

In one embodiment a salt may be present in concentrations from about 1 mM to 5M, such as about 1 mM, 5 mM, 10 mM, 20 mM, 30 mM, 40 mM, 50 mM, 100 mM, 200 mM, 300 mM, 400 mM, 500 mM, 750 mM, 1M, 1.5M, 2M, 2.5M, 3M, 3.5M, 4M, 4.5M, or 5M. In one embodiment a salt may be present in concentrations from about 1 mM to 2M, such as about 1 mM, 5 mM, 10 mM, 20 mM, 30 mM, 40 mM, 50 mM, 100 mM, 200 mM, 300 mM, 400 mM, 500 mM, 750 mM, 1M, 1.5M, or 2M. In another embodiment a salt may be present in concentrations from about 10 mM-1M, such as about 10 mM, 20 mM, 30 mM, 40 mM, 50 mM, 100 mM, 200 mM, 300 mM, 400 mM, 500 mM, 750 mM, or 1M. In another embodiment a salt may be present from about 10 mM-500 mM, such as about 10 mM, 20 mM, 30 mM, 40 mM, 50 mM, 100 mM, 200 mM, 300 mM, 400 mM, or 500 mM. In another embodiment a salt may be present from about 10 mM-200 mM, such as about 10 mM, 20 mM, 30 mM, 40 mM, 50 mM, 100 mM, or 200 mM.

Embodiments of the present invention may be compositions that comprise at least one RNA stabilizing substance, at least one RNA substance, and one or more chelating agent. These embodiments comprising one or more chelating agent may be any composition as described herein that may also comprise one or more chelating agent.

Non-limiting examples of one or more chelating agent that may be used, may include but are not limited to, one or more of the following: EDTA, HEDTA, EDDA, EGTA, DTPA, DTPMP, BAPTA, NOTA, DOTA, DMSA, nicotinanamine, EDDHA, EDDS, citrate, ATMP, polyaspartate, cyclam, cyclen, diethyldithiocarbamate, porphyrin, porphine, picolinate, malate, oxalate, iminodiacetic acid, β-thujaplicin, homocitrate, nitrilotriacetic acid, phenanthroline, 3-pyridylnicotinamide, 4-pyridylnicotinamide, gluconate, gallates, etidronate, dexrazoxane, deferoxamine, deferiprone, deferasirox, triethylenetetramine, tetramethylethylenediamine, or terpyridine, or mixtures, or combinations thereof.

In one embodiment a chelating agent may be present in concentrations from about 0.1 mM to 5M, such as about 0.1 mM, 0.5 mM, 1 mM, 5 mM, 10 mM, 20 mM, 30 mM, 40 mM, 50 mM, 100 mM, 200 mM, 300 mM, 400 mM, 500 mM, 750 mM, 1M, 1.5M, 2M, 2.5M, 3M, 3.5M, 4M, 4.5M, or 5M. In one embodiment a chelating agent may be present in concentrations from about 0.1 mM to 1M, such as about 0.1 mM, 0.5 mM, 1 mM, 5 mM, 10 mM, 20 mM, 30 mM, 40 mM, 50 mM, 100 mM, 200 mM, 300 mM, 400 mM, 500 mM, 750 mM, or 1M. In another embodiment a chelating agent may be present in concentrations from about 0.1 mM-500 mM, such as about 0.1 mM, 0.5 mM, 1 mM, 5 mM, 10 mM, 20 mM, 30 mM, 40 mM, 50 mM, 100 mM, 200 mM, 300 mM, 400 mM, or 500 mM. In another embodiment a chelating agent may be present from about 0.1 mM-100 mM, such as about 0.1 mM, 0.5 mM, 1 mM, 5 mM, 10 mM, 20 mM, 30 mM, 40 mM, 50 mM, or 100 mM. In another embodiment a chelating agent may be present from about 0.1 mM-10 mM, such as about 0.1 mM, 0.5 mM, 1 mM, 5 mM, or 10 mM.

In one embodiment a chelating agent may have a molecular weight between about 50 daltons-100,000 daltons. In one embodiment a chelating agent may have a molecular weight between about 50 daltons-50,000 daltons. In one embodiment a chelating agent may have a molecular weight between about 50 daltons-25,000 daltons. In one embodiment a chelating agent may have a molecular weight between about 50 daltons-10,000 daltons. In one embodiment a chelating agent may have a molecular weight between about 50 daltons-5,000 daltons. In one embodiment a chelating agent may have a molecular weight between about 50 daltons-1,000 daltons. In one embodiment a chelating agent may have a molecular weight between about 50 daltons-750 daltons. In one embodiment a chelating agent may have a molecular weight between about 50 daltons-500 daltons. In one embodiment a chelating agent may have a molecular weight between about 50 daltons-350 daltons. In one embodiment a chelating agent may have a molecular weight between about 100 daltons-750 daltons. In one embodiment a chelating agent may have a molecular weight between about 100 daltons-500 daltons. In one embodiment a chelating agent may have a molecular weight between about 100 daltons-350 daltons.

In one embodiment a combination comprising at least one RNA substance and at least one RNA stabilizing substance may be a liquid. As non-limiting examples, may be a solution, fluid, syrup, emulsion, or suspension and may also include liquid or solid carriers. As non-limiting examples the liquid viscosity may be in the range between 0.1 centipoise-100,000,000 centipoise at about 20-25° C. As non-limiting examples the liquid viscosity may be in the range between 0.1 centipoise-1,000,000 centipoise at about 20-25° C. As non-limiting examples the liquid viscosity may be in the range between 0.1 centipoise-100,000 centipoise at about 20-25° C. As non-limiting examples the liquid viscosity may be in the range between 0.1 centipoise-10,000 centipoise at about 20-25° C.

In one embodiment a combination comprising at least one RNA substance and at least one RNA stabilizing substance may be a solid. As non-limiting examples, may be a pellet, powder, or tablet, and may also include solid carriers.

In one embodiment a combination comprising at least one RNA substance and at least one RNA stabilizing substance may be a vapor or aerosol. As non-limiting examples, may be a gas, vapor, or aerosol, or suspension of particles or droplets suspended in one or more gases (such as, but not limited to, air, nitrogen, oxygen, carbon dioxide, or anesthetic gas) and may also include liquid or solid carriers.

In one embodiment, an RNA stabilizing substance may have a molecular weight between about 25-1,000,000 daltons. In one embodiment, an RNA stabilizing substance may have a molecular weight between about 25-500,000 daltons. In one embodiment, an RNA stabilizing substance may have a molecular weight between about 25-200,000 daltons. In one embodiment, an RNA stabilizing substance may have a molecular weight between about 25-100,000 daltons. In one embodiment, an RNA stabilizing substance may have a molecular weight between about 25-50,000 daltons. In one embodiment, an RNA stabilizing substance may have a molecular weight between about 25-25,000 daltons. In one embodiment, an RNA stabilizing substance may have a molecular weight between about 25-10,000 daltons. In one embodiment, an RNA stabilizing substance may have a molecular weight between about 25-5,000 daltons. In one embodiment, an RNA stabilizing substance may have a molecular weight between about 25-2,000 daltons. In one embodiment, an RNA stabilizing substance may have a molecular weight between about 25-1,000 daltons. In one embodiment, an RNA stabilizing substance may have a molecular weight between about 25-900 daltons. In one embodiment, an RNA stabilizing substance may have a molecular weight between about 25-800 daltons. In one embodiment, an RNA stabilizing substance may have a molecular weight between about 25-700 daltons. In one embodiment, an RNA stabilizing substance may have a molecular weight between about 25-600 daltons. In one embodiment, an RNA stabilizing substance may have a molecular weight between about 25-500 daltons. In one embodiment, an RNA stabilizing substance may have a molecular weight between about 25-400 daltons. In one embodiment, an RNA stabilizing substance may have a molecular weight between about 25-300 daltons. In one embodiment, an RNA stabilizing substance may have a molecular weight between about 25-250 daltons.

In one embodiment, an RNA stabilizing substance may have a molecular weight between about 50-1,000,000 daltons. In one embodiment, an RNA stabilizing substance may have a molecular weight between about 50-500,000 daltons. In one embodiment, an RNA stabilizing substance may have a molecular weight between about 50-200,000 daltons. In one embodiment, an RNA stabilizing substance may have a molecular weight between about 50-100,000 daltons. In one embodiment, an RNA stabilizing substance may have a molecular weight between about 50-50,000 daltons. In one embodiment, an RNA stabilizing substance may have a molecular weight between about 50-25,000 daltons. In one embodiment, an RNA stabilizing substance may have a molecular weight between about 50-10,000 daltons. In one embodiment, an RNA stabilizing substance may have a molecular weight between about 50-5,000 daltons. In one embodiment, an RNA stabilizing substance may have a molecular weight between about 50-2,000 daltons. In one embodiment, an RNA stabilizing substance may have a molecular weight between about 50-1,000 daltons. In one embodiment, an RNA stabilizing substance may have a molecular weight between about 50-900 daltons. In one embodiment, an RNA stabilizing substance may have a molecular weight between about 50-800 daltons. In one embodiment, an RNA stabilizing substance may have a molecular weight between about 50-700 daltons. In one embodiment, an RNA stabilizing substance may have a molecular weight between about 50-600 daltons. In one embodiment, an RNA stabilizing substance may have a molecular weight between about 50-500 daltons. In one embodiment, an RNA stabilizing substance may have a molecular weight between about 50-400 daltons. In one embodiment, an RNA stabilizing substance may have a molecular weight between about 50-300 daltons. In one embodiment, an RNA stabilizing substance may have a molecular weight between about 50-250 daltons.

Cellular Uptake Agents:

In one embodiment of the present invention a combination that comprises one or more RNA stabilizing substance and one or more RNA substance also comprises one or more substance either at least partially complexed with the RNA, and/or one or more substance that at least partially encapsulates the RNA, to promote the RNA's ability to enter cells (herein referred to as cellular uptake agents), example substances being, including but not limited to, lipids, polymers, polymeric materials, zwitterionic polymers, zwitterionic lipids, ionizable polymers, ionizable lipids, cationic polymers, cationic lipids, amino-lipids, cholesterols, cationic detergents, zwitterionic detergents, ionizable detergents, non-ionic detergents, detergents, polyethylenimine (PEI), polyplexes, polyamines, lipid nanoparticles, detergent micelles, micelles, liposomes, nanoliposomes, lipoparticles, nanolipoparticles, dendrimers, particles, nanoparticles, lipid membranes, lipid micelles, lipid bilayers, or membrane vesicles. Examples of cell entry may include, but are not limited to, fusion with the cellular membrane, endocytosis, pinocytosis, phagocytosis, passive diffusion, active diffusion, osmotic diffusion, facilitated diffusion, diffusion, hole formation, direct microinjection, electroporation, ultrasound, energy induced, electricity induced, electric field induced, or similar mechanisms to deliver the RNA substance to, including but not limited to, a cell, eukaryotic cell, prokaryotic cell, plant cell, fungal cell, plant, bacteria, fungus, insect, organ, tissue, animal, or vertebrate animal, including but not limited to a human, by entering cells.

As used herein, cellular uptake agents means substances that promote RNA's ability to enter cells and may include, but are not limited to, substances that may at least partially bind to or at least partially complex with RNA or substances that may at least partially encapsulate RNA.

Cellular uptake agents are known art when used in aqueous RNA solutions and may also be referred to as gene delivery agents, transfection agents, cellular delivery agents, intracellular delivery agents, or complexation agents. The present invention uses cellular uptake agents in the novel configuration of one or more cellular uptake agent with one or more RNA stabilizing substance and one or more RNA substance. At least one or more cellular uptake agent may be combined with at least one or more RNA stabilizing substance and at least one or more RNA substance either in advance and stored together or stored separately, such as in a two-chamber container, and combined close to the time of administration.

One embodiment of the present invention includes a composition comprising a combination of one or more RNA substance, one or more RNA stabilizing substance, and one or more cellular uptake agent.

One embodiment of the present invention includes a combination comprising a mixture of one or more RNA substance, one or more RNA stabilizing substance, and one or more cellular uptake agent.

Embodiments of the present invention that comprise one or more RNA substance, one or more RNA stabilizing substance, and one or more cellular uptake agent may include combining, such as by mixing, one or more RNA substance with one or more RNA stabilizing substance and one or more cellular uptake agent.

In one embodiment of the present invention a composition comprising a combination of one or more RNA substance, one or more RNA stabilizing substance, and one or more cellular uptake agent produces a mixture with at least one or more RNA substance, at least one or more RNA stabilizing substance, and at least one or more cellular uptake agent.

In one embodiment of the present invention a combination comprising one or more RNA substance, one or more RNA stabilizing substance, and one or more cellular uptake agent produces a mixture with at least one or more RNA substance, at least one or more RNA stabilizing substance, and at least one or more cellular uptake agent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As used herein, chamber means a compartment capable of containing at least one of a solid, powder, liquid, aerosol, or gas that may be sealed and later allow at least some of its contents to be at least one of partially delivered, removed, emptied, dispensed, opened, accessed, or penetrated. As a non-limiting example, a chamber may be at least one of, including but not limited to, bottles, containers, vials, tubes, jars, syringes (including prefilled syringes), blisters, capsules, tablets, cartridges, inhalers, packets, pods, or other packages that may hold a solid, powder, liquid, aerosol, or gas. As a non-limiting example, chambers may contain up to 100 kg of stabilized RNA composition, or may contain up to 1 kg of stabilized RNA composition, or may contain up to 10 g of stabilized RNA composition, or may contain up to 100 mg of stabilized RNA composition.

In one embodiment of the present invention a chamber may comprise one or more RNA substance with one or more RNA stabilizing substance. In one embodiment the chamber may be a vial. In one embodiment the chamber may be a prefilled syringe.

In one embodiment of the present invention a chamber may comprise one or more RNA substance with one or more RNA stabilizing substance and one or more cellular uptake agent. In one embodiment the chamber may be a vial. In one embodiment the chamber may be a prefilled syringe.

In one embodiment of the present invention a chamber may comprise one or more RNA substance with one or more RNA stabilizing substance, one or more cellular uptake agent and one or more buffering agent. In one embodiment the chamber may be a vial. In one embodiment the chamber may be a prefilled syringe.

In one embodiment of the present invention a chamber may comprise one or more RNA substance with one or more RNA stabilizing substance, one or more cellular uptake agent, one or more buffering agent and one or more salt. In one embodiment the chamber may be a vial. In one embodiment the chamber may be a prefilled syringe.

In one embodiment of the present invention a chamber may comprise one or more RNA substance with one or more RNA stabilizing substance, one or more cellular uptake agent, one or more buffering agent, one or more salt, and one or more chelating agent. In one embodiment the chamber may be a vial. In one embodiment the chamber may be a prefilled syringe.

In one embodiment of the present invention, each component included in a composition comprising one or more RNA substance, one or more RNA stabilizing substance, and one or more cellular uptake agent, may be stored separately, such as in a kit, or such as individually or as mixtures of one or more substance, and then combined later to produce a composition comprising one or more RNA substance, one or more RNA stabilizing substance, and one or more cellular uptake agent.

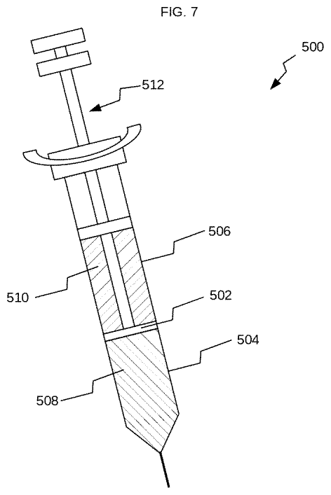

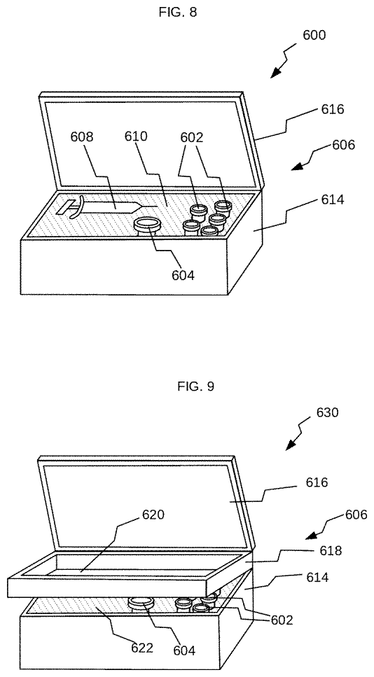

An example of such a kit 600 is shown in FIG. 8. As shown, the kit 600 includes one or more component vials 602 and one or more mixing/dispensing vials 604 contained in a package 606, such as a hinged box container. Each of the component vials 602 may contain one or more components of the composition as described herein. For example, each component may be provided in a separate vial 602 or certain compatible components may be combined in a single vial 602 with other components provided individually or in combination in other vials 602.

The components can then be mixed in mixing/dispensing vial 604, for example, immediately prior to use, in order to minimize RNA degradation. In this regard, the components from the vials 602 may be transferred into the vial 604, e.g., poured or injected into the vial 604 using a syringe 608, and then shaken or otherwise mixed. The components in the vials 602 may be premeasured and may provide a single unit or dose of the composition or multiple units/doses. Alternatively, the components from the vials 602 may be measured and combined by skilled workers. Optionally, e.g., in the case of vaccines, one or more syringes 608 may be provided in the kit 600 for drawing the composition from the vial 604 and administering to subjects. The vials 602 and 604 and syringes 608 may be secured by packing material 610 such as a foam material.

As described herein, many compositions, components, or combinations of components in accordance with the present invention can be stored without requiring extremely low temperatures. In cases where cold storage is required, the kit 600 can be transported in a cold storage unit or cold storage vehicles. The packaging 606 may be formed from materials suitable to withstand such cold storage such as various plastics or metals. In such cases, the vials 602, 604 and syringes 608 (if provided in the kit 600) may be formed from materials selected to withstand cold storage.

Although the kit is shown as including vials 602, 604 and syringes 608 for purposes of illustration, it will be appreciated that the components may be provided in other forms, e.g., non-liquid forms, and the composition may be provided for purposes other than vaccination. Accordingly, while a kit including some or all of the components of a composition in accordance with the present invention is useful and convenient, the nature of the kit can vary from the kit shown.

As a non-limiting example, vials 602 may comprise a concentrated composition comprising at least one RNA substance and at least one RNA stabilizing substance that after being mixed with at least one diluent is suitable for use, such as suitable for injection. In this embodiment the diluent may or may not be part of kit 600.

In another embodiment packaging 606 may comprise vials 602 that may be ready for use and packaging 606 provides a package for uses comprising at least one of as storage and transport and maintaining a desired environment for vials 602. Packaging 606 may comprise a cooling pack (not shown) that at least partly offsets thermal energy transferred from the storage and transport environment to the inside of package 606 where at least some of vials 602 are desired to experience a maximum target temperature.

In one embodiment kit 600 may comprise a cooling substance (not shown) that maintains the temperature of at least one of vials 602 at no more than a maximum target temperature. As a non-limiting example, the maximum target temperature may be about 4° C. or less. As a non-limiting example, the maximum target temperature may be about 20° C. or less. As a non-limiting example, a cooling substance that maintains the maximum target temperature or less may undergo a phase change to maintain the maximum target temperature. Non-limiting examples of cooling substances that may be used to maintain the maximum target temperature are solid phase water that may change to liquid phase water, solid phase carbon dioxide that may change phase to vapor phase carbon dioxide, or liquid phase nitrogen that may change from liquid phase to vapor phase nitrogen. Other non-limiting examples of cooling substances that may be used to maintain the maximum target temperature are refrigerants circulated through heat exchangers used in compression refrigeration (for example, haloalkane refrigerants or hydrocarbons), adsorption refrigeration, or absorption refrigeration. As a non-limiting example, the maximum target temperature or less may be maintained using at least one substance that does not undergo phase change. As non-limiting examples of cooling substances that may be used to maintain the maximum target temperature or less that do not undergo phase changes are thermoelectric coolers of which Peltier devices that may be incorporated into one or more walls of package 606 are non-limiting examples.

As non-limiting examples the cooling substance used to maintain the maximum target temperature or less may be cooling packs comprising a package containing one or more substances that maintain the target temperature or less. A non-limiting example of a cooling pack is a plastic package containing water that may be cooled to be at or less than the target temperature including being cooled to produce at least some water ice in the package. As non-limiting examples the cooling substance may comprise water and at least one substance that when mixed with water increases the viscosity. As non-limiting examples, the substance mixed with water to increase viscosity may be hydroxyethyl cellulose, sodium polyacrylate, or vinyl-coated silica gel. As non-limiting examples, the cooling substance used to maintain the maximum target temperature or less may comprise a package containing water and a viscosity increasing material and the viscosity increasing material may comprise at least one of hydroxyethyl cellulose, sodium polyacrylate, or vinyl-coated silica gel. As non-limiting examples, the cooling substance used to maintain the maximum target temperature or less (as a non-limiting example, comprising a package, water, and a viscosity increasing substance) the package may be placed in an environment that is colder than the maximum target temperature before use to maintain the target temperature. As a non-limiting example, the substance may comprise a flexible plastic package, water, and a viscosity increasing substance and the package with water and a viscosity increasing material may be placed in a freezer to cool the package before it is used to maintain the target temperature or less. As non-limiting examples, frozen gel-packs may be used to maintain the maximum target temperature or less.

As a non-limiting example, kit 600 may comprise at least one substance comprised of two or more materials that when mixed cause an endothermic chemical reaction that leads to absorbing energy from the surroundings to maintain the temperature of at least one vial 602 at a maximum target temperature or less. As a non-limiting example, the substance maintaining the temperature at the maximum target temperature or less may comprise a flexible plastic package containing at least one endothermic producing compound and also contain a sealed package holding water such that the inner bag of water can be broken by a user to cause the water to mix with at least one endothermic producing compound. As a non-limiting example, a user may break the inner bag of water by squeezing the package, allowing the water to mix with at least one endothermic producing compound to produce endothermic reactions, followed by the user placing the package in kit 600 to maintain the temperature of at least on vial 602 at the maximum target temperature. As non-limiting examples, the endothermic producing compound may be at least one of ammonium nitrate, calcium ammonium nitrate, or urea. As a non-limiting example, kit 600 may comprise a substance that maintains the temperature of at least one vial 602 at a maximum target temperature or less in which the temperature maintaining substance comprises water and an endothermic producing compound in which the water and endothermic producing compound may be separated until use and be available for mixing by having the water and endothermic producing compound in a package that comprises an inner package that holds water separated from endothermic producing compounds until the materials are mixed.

As a non-limiting example, one embodiment of the present invention is a kit comprising at least one RNA substance and at least one RNA stabilizing substance in which such kit has a package 606 with inside volume that allows maintaining a maximum target temperature or less by placing at least one cooling pack. Non-limiting examples of cooling packs include endothermic cooling packs or gel-packs that have been cooled such as by having been in a freezer or been in an environment with a cold substance of which non-limiting examples include dry ice or water ice or water ice mixed with brine.

A non-limiting example embodiment of kit 600 comprises component vials 602 that may contain one or more components of the composition as described herein in which at least one component is an RNA substance and at least one component is an RNA stabilizing substance (which may be an aprotic substance) and a substance that maintains the maximum target temperature of at least one of the vials 602 to about 4° C. or less for at least one hour. In a non-limiting embodiment the kit may maintain the temperature of at least one vial at about 20° C. or less for at least 24 hours. As another non-limiting embodiment, kit 600 may maintain the temperature of at least one vial at about 20° C. or less for at least 60 hours.

Non-limiting embodiments of kits comprise at least one RNA substance, at least one RNA stabilizing substance, a package, and at least one of a temperature recording device and a location tracking device. A non-limiting example of temperature recording devices may comprise a temperature sensitive substance that irreversibly changes to indicate when a specified temperature has been exceeded, such as by causing a chemical of physical change visible to a user. As non-limiting example of temperature recording devices are devices comprising temperature measuring and logging devices that comprise one or more electronic component that may be thermocouples, thermistors, or other temperature sensing element. Non-limiting examples of temperature recording devices are devices with at least one electronic communication component comprising a visual display or a radio-frequency transmitter and may comprise wi-fi or cellular communication capability. Non-limiting examples of location tracking devices may comprise GPS or inertial location devices with at least one electronic communication component comprising a visual display or a radio-frequency transmitter and may comprise wi-fi or cellular communication capability.

FIG. 9 illustrates a non-limiting example embodiment of the present invention as kit 630 that comprises component vials 602 that may contain one or more components of the composition as described herein in which at least one component is an RNA substance and at least one component is an RNA stabilizing substance in package 606 that may have cooling substance compartment 618 that has cooling substance chamber 620 that may contain a cooling substance with cooling substance chamber 620 at least partly separated from component compartment 622 containing at least one of vials 602. Package 606 comprises container box base 614, container box cover 616, and cooling substance compartment 618. Cooling substance compartment 618 may form at least part of the cover of container box base 614 holding one or more vials 602. Container box cover 616 may form at least part of a cover for cooling substance compartment 618. Cooling substance compartment 618 may have cooling substance chamber 620 into which may be placed a cooling substance of which a non-limiting example is a cooling pack. The non-limiting example kit illustrated in FIG. 9 has cooling substance chamber 620 separate from component compartment 622 containing vials 602, a configuration that allows replacing cooling substance without opening the compartment containing other components of the kit, such as vials 602, providing secure storage of those components when cooling substances are added or replenished. FIG. 9 illustrates separation of at least some vials 602 from cooling substances using cooling substance compartment 618. As a non-limiting alternative example, the separation of cooling substances from a compartment containing at least of vials 602 may be one or more packages such as bags or boxes used with cooling substances which, as non-limiting examples, may be packs containing ice, cooling gel packs, or endothermic cooling packs.

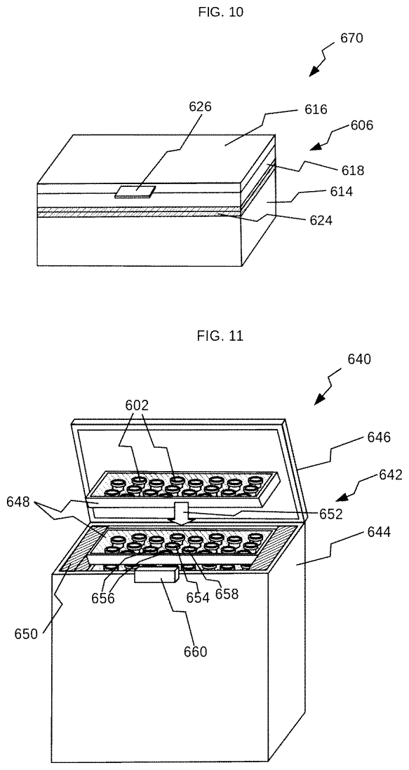

FIG. 10 illustrates that a kit comprising at least one RNA substance and at least one RNA stabilizing substance may comprise a security measure 624 that indicates whether the kit has been opened. As a non-limiting example, security measure 624 may be an adhesive sealing strip that changes appearance when removed or tampered with, such as by having at least one of a color or pattern change. Security measure 624 may also help fix one part of a kit comprising an RNA substance and an RNA stabilizing substance to another part of the kit. A non-limiting example is two parts of kit package 606 where security measure 624 may be a band with adhesive that helps fix container box base 614 to cooling substance compartment 618. As non-limiting examples, security measure 624 may comprise an adhesive strip that attaches in part to container box base 614 and in part to cooling substance compartment 618. As another non-limiting example, security measure 624 may be used with kits to help secure a container box cover to a container base or cooling substance compartment. In other embodiments security measure 624 may comprise a mechanical lock.

FIG. 10 illustrates that a kit comprising at least one RNA substance and at least one RNA stabilizing substance may comprise a latch 626 that reversibly fixes at least one component of the kit to another component. As non-limiting examples, latch 626 may reversibly fix container box cover 616 to cooling substance compartment 618 as shown in FIG. 10. Other non-limiting latching configurations may also be used, such as non-limiting examples where latch 626 reversibly fixes container box cover 616 to container box base 614 and where a latch reversibly fixes cooling substance compartment 618 to container box base 614. More than one latch may be used to reversibly fix components of the kit together. Non-limiting examples of latches are over-center toggle latches, magnetic latches, or hasps that may be locked or fixed with tamper-evident indicators such as wires.

FIG. 11 illustrates container 640 as a non-limiting example of a shipping and storage container that may be used, for example, to package chambers with compositions comprising at least one RNA substance and at least one RNA stabilizing substance. As a non-limiting example, the chambers may comprise vials or pre-filled syringes that may comprise an RNA substance, an RNA stabilizing substance and a cellular uptake agent. As non-limiting examples, FIG. 11 illustrates the chambers as vials 602 and for consistency in this description chambers are referred to as vials 602. Container 640 may comprise component vials 602 that may contain one or more components of the composition as described herein in which at least one component is an RNA substance and at least one component is an RNA stabilizing substance in package 642 that may have component vials 602 in at least one carrier 648 which, as a non-limiting example, may be a tray. Non-limiting alternative embodiments may comprise vials 602 containing ready to use mixtures comprising at least one RNA substance and at least one RNA stabilizing substance or concentrated mixtures comprising at least one RNA substance and at least one RNA stabilizing substance that when diluted are suitable for use. As a non-limiting example, a diluent may be a substance comprising water or other biocompatible substance.

Package 642 comprises container box 644 holding one or more carriers 648. Package 642 may comprise at least one opening at least partly reversibly closable with at least one cover 646. Cover 646 and the associated opening may be located at or near the top of package 642 or cover 646 and the associated opening may be located on one or more sides or the bottom of package 642.

In one non-limiting embodiment at least one of container box 644 and cover 646 may comprise at least one material having thermal conductivity less than about 0.2 W/m ° K. In one non-limiting embodiment, at least one of container box 644 and cover 646 may comprise at least one material having thermal conductivity less than about 0.1 W/m ° K. In one non-limiting embodiment at least one of container box 644 and cover 646 may comprise at least one material having thermal conductivity less than about 0.05 W/m ° K. In one non-limiting embodiment at least part of at least one of container box 644 and cover 646 may have an overall heat transfer coefficient less than about 20 W/m²° K. In one non-limiting embodiment at least part of at least one of container box 644 and cover 646 may have an overall heat transfer coefficient less than about 10 W/m²° K. In one non-limiting embodiment at least part of at least one of container box 644 and cover 646 may have an overall heat transfer coefficient less than about 5 W/m²° K. In one non-limiting embodiment at least part of at least one of container box 644 and cover 646 may have an overall heat transfer coefficient less than about 2 W/m²° K. In one embodiment package 642 has at least one coolant chamber 650 that may contain a cooling substance with coolant chamber 650 at least partly separated from at least one of carriers 648 containing at least one of vials 602. As non-limiting examples, one or more coolant chamber 650 may be at least partly on or near one or more sides of container box 644 as illustrated in FIG. 11 or one or more cooling chamber 650 may be at least partly on or near the bottom of container box 644 or one or more cooling chamber 650 may be at least partly on or near the top of container box 644 one or more cooling chamber 650 may be at least partly part of or attached to cover 646. Package 642 may comprise container box 644, container box cover 646, and cooling chamber 650. More than one carrier 648 may be placed in container box 644. In a non-limiting example, carriers 648 may be placed to form a vertical stack of trays by placing trays one on top of the other, possibly with at least one separator (not shown) between carriers 648, as illustrated by arrow 652 indicating a vertical insertion of carriers 648 into container box 644.

Carriers 648 may be made from polymeric substances using known methods with example methods being injection molding, blow-molding, pressure forming, casting, machining, or vacuum forming. In one non-limiting embodiment carriers 648 may be formed with individual compartments (not shown) for vials 602. As a non-limiting example, such compartments may be sized to at least partly separate vials or to at least partly secure vials 602. Such at least partial separation or at least partial securing may reduce impact, vibrations, or contacting vials to reduce the possibility of breakage or other damage during shipping, transport, storage or other movement of vials 602.

Carriers 648 may be any size convenient for shipping and use. As non-limiting examples, carriers 648 may have a length or width of between about 10 cm and 100 cm and carriers may have a length or width between about 30 cm and 40 cm. As non-limiting examples, carriers 648 may have a length or width between about 10 cm and about 20 cm.

In one embodiment, carriers 648 may nest with each other or may nest with vials 602 to facilitate securing vials in the carriers. For example, the bottoms of carriers 648 may have recesses that mate with the tops of vials 602 such that when a carrier is removed the absence of the bottom recess in the removed carrier reveals a section of the vial beneath the carrier that may be grasped but when the carrier is in place the recess helps maintain the positions of vials 602 during shipping and storage. It will be appreciated that although carriers 648 are illustrated and described as holding vials 602 vertically that other orientations may also be employed, such as carriers being inserted vertically and holding vials 602 vertically or holding vials 602 in a horizontal orientation. As a non-limiting example, the compartments of carriers 648 may be configured to have interference fits with vials 602. As a non-limiting example of an interference fit, at least a portion of the openings of compartments may be smaller than a dimension of vials 602 so that vials 602 press into the openings to be removably secured in compartments of carriers 648. As a non-limiting example, trays may have compartments that hold vials 602 with the axis of the vials parallel to the plane of carrier 648 such that when carrier 648 is inserted into container box 644 with carriers 648 at least partially vertically oriented the vials 602 are positioned with the axis of the vials being at least partially vertically oriented. As a non-limiting example, when carriers 648 are oriented at least partially vertically the opposing face of the adjacent carrier may be shaped to allow a single carrier to be withdrawn from container box 644 without adjusting the positions of other carriers 648. Alternatively, carriers may be positioned horizontally with the opening associated with cover 646 positioned on the side of container box 644. It will be appreciated that chambers other than vials may be used to contain RNA substances and RNA stabilizing substances and other materials as described herein to have mixtures of RNA substances and RNA stabilizing substances and such other chambers may be substituted for at least some of vials 602. When chambers other than vials are present in carriers 648 the carriers may be configured to both provide access to chambers when carriers are removed and to help maintain positions of chambers during shipping and storage when carriers are in place, similar to as described above for vials.

As a non-limiting example, at least one carrier 648 may have vials arranged to efficiently pack vials 602 to save space compared to less efficient packing arrangements. A non-limiting example of efficiently packing vials 602 is a hexagonal cell surrounding a central vial. FIG. 11 illustrates a hexagonal cell with central vial 654 surrounded by six other approximately equally spaced vials with examples of surrounding vials being vials 656 and 658. In one non-limiting embodiment, at least one carrier 648 may have compartments into which vials 602 may be placed to efficiently pack them with one non-limiting example being at least one carrier 648 having compartments that may hold at least seven vials in an approximately hexagonal pattern with at least one vial surrounded by six other vials that are approximately equally spaced from the central vial and from each other with the spacing between centers being between about 5 percent and 50 percent greater than the diameter of vials 602.

In one non-limiting embodiment, package 642 may have a latch 660 that reversibly holds cover 646 in a closed position on box 644, with FIG. 11 illustrating as a non-limiting example latch 658 attached to box 644 and capable of reversibly securing cover 646 to close the opening of box 644.

As a non-limiting example, container 640 may comprise least one of a temperature recording device and a location tracking device as described earlier. As a non-limiting example, container 640 may comprise a security measure as described earlier.

In non-limiting embodiments of the present invention package 642 may be cooled using phase change substances of the types described earlier. As non-limiting examples, vials 602 may comprise at least on RNA substance and at least RNA stabilizing substance and one or more substances other than dry ice may be used as the cooling substance in one or more compartment 650 to maintain a temperature substantially warmer than the −78° C. for which dry ice is used.

Non-limiting embodiments of the present invention may comprise RNA substances and RNA stabilizing substances and water ice as a cooling substance to simplify shipping and storage of RNA substances by not using dry ice. When dry ice is used as the cooling substance the dry ice sublimates to offset the energy entering by thermal conduction into the chamber containing RNA substances. The thermal energy transfer rate (heat transfer rate) may be approximated by the expression Q=U×A×ΔT where Q is the heat transfer rate, U is the overall heat transfer coefficient, and ΔT is the temperature difference between the inside of the package containing the RNA substance and the environment. The heat transfer rate is proportional to that temperature difference. ΔT=Tₑ−Tᵥ where Tₑ=the temperature of the outside environment, such as 20° C. and Tᵥ=the target temperature at which vials of RNA substance may be maintained. Dry ice may be used when low Tᵥ is required, such as −78° C., leading to ΔT=20−(−78), equal to 98° C. As a non-limiting example, using RNA stabilizing substances and Tᵥ=4° C. leads to ΔT=20−4, equal to 16° C. For this example ΔT with dry ice is over six times greater than when using RNA stabilizing substances. The result is that the thermal energy transfer rate is over six times greater when RNA stabilizing substances are not used and dry ice is required. The cooling capacity of dry ice comes from it sublimating at −78° C. at which 199 kJ/kg of energy is absorbed. The density of dry ice is about 1,562 kg/m³ so each liter of dry ice will absorb about 311 kJ. The cooling capacity of water ice comes from it melting at 0° C. at which 334 kJ/kg of energy is absorbed. Accounting for the density of water ice (917 kg/m³) each liter of water ice will absorb about 306 kJ. These results demonstrate that a shipping container designed to hold a specific volume of cooling substance will have about the same amount of cooling capacity for both dry ice and water ice. However, with the RNA stabilizing substance and using Tv=4° C. the rate of heat gain with water ice is about one-sixth that which occurs when using dry ice so the shipping container with water ice will maintain the target vial temperature much longer. Alternatively, a shipping package comprising an RNA substance and an RNA stabilizing substance may be smaller when cooled with water ice than a shipping package that uses dry ice and no RNA stabilizing substance.

The preferred thickness of insulating walls of package 642 is approximately equal to the thickness of cooling chamber 650. The time that a cooling substance can cool a package is

$$τ = \frac{C}{Q}$$

where C=cooling capacity of cooling substance (Joules), Q=thermal energy transfer rate (J/hr).

$$C = {t}_{c} A H$$

where t_{c}=thickness of the cooling substance, A=area of face of cooling material against, for example, a wall of the package. H=energy absorbing capacity per unit volume of cooling substance.

$$C = \frac{k A Δ T}{{t}_{i}}$$

where k=thermal conductivity of side wall, tᵢ=thickness of the insulated side wall, A=area of side wall (assumed to be the same as area of the cooling substance against that wall), ΔT=temperature difference between the cooling substance and the environment.

After substitution obtain

$$τ = \frac{{t}_{c} H {t}_{i}}{k Δ T}$$

know that that the total thickness is the thickness of the insulation thickness plus the thickness of the cooling substance, therefore the thickness of the cooling substance is the difference between the total thickness and insulation thickness.

$${t}_{c} = {t}_{t} - {t}_{i}$$

After substitution obtain

$$τ = \frac{( {t}_{i} {t}_{i} - {t}_{i}^{2} ) H}{Δ Tk}$$

Take derivative and set to 0 to find optimum and solve for optimum insulation

$$\frac{d τ}{{dt}_{i}} = \frac{( {t}_{i} - 2 {t}_{i} ) H}{Δ Tk}$$

thickness

Optimum insulation thickness is about one-half of total thickness of the insulation and the cooling substance, i.e., the optimum insulation thickness is about the same

$${t}_{i} = \frac{1}{2} {t}_{i}$$

thickness as the cooling substance thickness, and is independent of cooling substance, temperature difference, and insulation thermal conductivity.

In one embodiment of the present invention a composition comprising one or more RNA substance, one or more RNA stabilizing substance, and one or more cellular uptake agent may be at least partially biocompatible.

In one embodiment of the present invention comprising a combination of one or more RNA substance, one or more RNA stabilizing substance, and one or more cellular uptake agent, may be at least partially biocompatible.

In another embodiment at least one or more cellular uptake agent may be combined with at least one or more RNA stabilizing substance, as described herein, and at least one or more RNA substance either in advance and stored together or stored separately, such as in a two-chamber container, and combined close to the time of administration.

### Examples of Cellular Uptake Agents:

In one embodiment a cellular uptake agent may comprise, but is not limited to, at least one or more of the following: a lipid, polymer, polymeric material, zwitterionic polymer, zwitterionic lipid, ionizable polymer, ionizable lipid, cationic polymer, cationic lipid, amino-lipid, cholesterol, cationic detergent, zwitterionic detergent, ionizable detergent, non-ionic detergent, detergent, polyethylenimine (PEI), polyplexes, polyamines, lipid nanoparticles, detergent micelles, micelles, liposomes, nanoliposomes, lipoparticles, nanolipoparticles, dendrimers, particles, nanoparticles, lipid membrane, lipid micelle, lipid bilayer, or membrane vesicles, or derivatives, mixtures, or combinations thereof.

In another embodiment a cellular uptake agent may comprise, but is not limited to, at least one or more of the following surrounding an aqueous core or a hydrophobic core: a lipid, polymer, polymeric material, zwitterionic polymer, zwitterionic lipid, ionizable polymer, ionizable lipid, cationic polymer, cationic lipid, amino-lipid, cholesterol, cationic detergent, zwitterionic detergent, ionizable detergent, non-ionic detergent, detergent, polyethylenimine (PEI), polyplexes, polyamines, lipid nanoparticles, detergent micelles, micelles, liposomes, nanoliposomes, lipoparticles, nanolipoparticles, dendrimers, particles, nanoparticles, lipid membrane, lipid micelle, lipid bilayer, or membrane vesicles, or derivatives, mixtures, or combinations thereof.

As used herein, PEG is polyethylene glycol.

As used herein, a PEG lipid is a lipid modified with or conjugated to polyethylene glycol.

In another embodiment a cellular uptake agent may comprise, but is not limited to one or more the following: phospholipids, sterols, cholesterol, phospholipid-free lipid particle, non-cationic lipid, cholesterol-free lipid particle, noncyclic phosphate containing lipids, lipid conjugates, PEG-conjugated lipids, PEG-lipid conjugates, ATTA-lipid conjugates, cationic-polymer-lipid conjugates, PEG coupled to dialkyloxypropyls, PEG coupled to diacylglycerol, PEG coupled to phospholipids such as phosphatidylethanolamine, PEG conjugated to ceramides, or PEG conjugated to cholesterol, or derivatives, mixtures, or combinations thereof.

In some embodiments, a cellular uptake agent may comprise a polyethylene glycol-lipid, PEG or PEG-modified lipids (also known as PEGylated lipids), including but not limited to, at least one or more of the following: PEG-modified phosphatidylethanolamines, PEG-modified phosphatidic acids, PEG-modified ceramides, PEG-modified dialkylamines, PEG-modified diacylglycerols, or PEG-modified dialkylglycerols, or derivatives, mixtures, or combinations thereof.

In another embodiment a cellular uptake agent may comprise, including but not limited to, at least one or more of the following hydrophilic polymers substituted for or used in combination with PEG as described herein: polyvinylpyrrolidone, polymethyloxazoline, polyethyloxazoline, polyhydroxypropyl methacrylamide, polymethacrylamide or polydimethylacrylamide, polylactic acid, polyglycolic acid, or derivatized celluloses such as hydroxymethylcellulose or hydroxyethylcellulose, or derivatives, combinations, or mixtures thereof.

In another embodiment a cellular uptake agent may comprise, including but not limited to, at least one or more of the following: cholesterol, fecosterol, sitosterol, ergosterol, campesterol, stigmasterol, brassicasterol, tomatidine, tomatine, ursolic acid, alpha-tocopherol, corticosteroids, prednisolone, dexamethasone, prednisone, or hydrocortisone, or derivatives, combinations, or mixtures thereof.

In some embodiments, a cellular uptake agent may comprise a polymer, cationic polymer, cationic or polycationic compounds, or cationic polysaccharides, for example chitosan, polybrene, or polyethyleneimine (PEI), or derivatives, combinations, or mixtures thereof.

In some embodiments, a cellular uptake agent may comprise, including but not limited to, one or more of cationic peptides or proteins, cell penetrating peptides, basic polypeptides, basic amino acids or their derivatives, cationic dendrimers, polyamines, polyamine sugars, amino polysaccharides, oligofectamine, modified polyaminoacids, aminoacid-polymers, reversed polyamides, modified polyethylenes, modified acrylates, modified amidoamines, modified polybetaaminoester, dendrimers, polypropylamine dendrimers, poly(amidoamine) PAMAM based dendrimers, polyimines, poly(ethyleneimine), poly(propyleneimine), polyallylamine, polylysine, polyornithine, poly/lysine/ornithine, poly(propylene imine), poly(vinyl amine), poly(2-aminoethyl methacrylate), sugar backbone based polymers, cyclodextrin based polymers, dextran based polymers, chitosan, or silane backbone based polymers, or derivatives, combinations, or mixtures thereof.

In some embodiments, a cellular uptake agent may be linear, branched, or dendrimeric in structure.

In another embodiment a cellular uptake agent may comprise, including but not limited to, at least one or more of the following: polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, or polyarylates, or derivatives, combinations, or mixtures thereof.

In a non-limiting example, a cellular uptake agent comprised of a polymer may comprise, including but not limited to, one or more of the following: poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene or polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) or copolymers or mixtures thereof, polydioxanone or its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), or polyglycerol, or derivatives, combinations, or mixtures thereof.

Non-limiting examples of cellular uptake agents that may be suitable for use with the RNA stabilizing substances of the present invention are described in US Patent Application Pub. No. US 2020/0383922 A1, incorporated herein by reference, as cationic or polycationic compounds that may be used as transfection or complexation agents.

Non-limiting examples of cellular uptake agents that may be suitable for use with the RNA stabilizing substances of the present invention are described in U.S. Pat. Nos. 10,702,600 and 10,933,127, incorporated herein by reference, as nanoparticle formulations, cationic lipid nanoparticles, nanoparticles, liposomes, lipoplexes, lipid nanoparticles (LNPs), lipids, cationic lipids, ionizable lipids, PEG lipids, structural lipids, neutral lipids, non-cationic lipids, therapeutic nanoparticles, polymeric material, polymer-vitamin conjugate, block co-polymer or tri-block co-polymer, surface altering agents, or cationic or polycationic compounds.

Non-limiting examples of cellular uptake agents that may be suitable for use with the RNA stabilizing substances of the present invention are described in U.S. Pat. Nos. 8,058,069 and 9,364,435, incorporated herein by reference, as lipid particles, stable nucleic acid-lipid particle (SNALP), lipids, lipid conjugates, amphipathic lipids, neutral lipids, non-cationic lipids, anionic lipids, cationic lipids, hydrophobic lipids, or sterols.

Non-limiting examples of cellular uptake agents that may be suitable for use with the RNA stabilizing substances of the present invention are described in PCT Patent Application Pub. No. WO 2021/156267 A1, incorporated herein by reference, as polymeric carriers, lipidoids or cationic lipidoids, lipid nanoparticles (LNPs), liposomes, lipoplexes, nanoliposomes, lipids, cationic or polycationic lipids, neutral lipids, ionizable lipids, polymer conjugated lipids, cationic or polycationic compounds, cationic or polycationic polymers, cationic or polycationic polysaccharides, cationic or polycationic proteins, or cationic or polycationic peptides.

Non-limiting examples of cellular uptake agents that may be suitable for use with the RNA stabilizing substances of the present invention are described in U.S. Pat. No. 8,367,628, incorporated herein by reference, as lipids, amphoteric lipids, amphoteric liposomes, amphoteric liposomal mixtures, liposomal mixtures, sterols, cationic lipids, chargeable cationic lipids, chargeable anionic lipids, stable anionic lipids, neutral lipids, or mixtures of lipid components with amphoteric properties.

Non-limiting examples of cellular uptake agents that may be suitable for use with the RNA stabilizing substances of the present invention are described in US Patent Application Pub. No. US 2021/0260097 A1, incorporated herein by reference, as nanoparticles, lipid nanoparticles (LNPs), lipids, cationic or ionizable lipids, anionic lipids, neutral lipids, amphipathic lipids, PEGylated lipids, or structural lipids.

Non-limiting examples of cellular uptake agents that may be suitable for use with the RNA stabilizing substances of the present invention are described in US Patent Application Pub. No. US 2021/0261627 A1, incorporated herein by reference, as cationic or polycationic compounds, polymeric carriers, cationic polysaccharides, cationic lipids, polymers, cationic or polycationic polymers, copolymers, blockpolymers, or cationic or polycationic proteins or peptides, which may be used as transfection or complexation agents.

### Containers and Mixing

Containers, such as syringes or vials, may hold a combination of at least one or more RNA stabilizing substance and at least one or more RNA substance and the containers may also contain at least one or more cellular uptake agents that are kept separate from the RNA stabilizing substance and the RNA substance until close to the time of use.

As a non-limiting example, a vial may hold a combination of materials comprising at least one or more RNA substance and at least one or more RNA stabilizing substance that is ready for injection after removal from the vial such as by withdrawal using a syringe and needle. As a non-limiting example, a vial may hold a combination of materials that is a concentrate for injection comprising at least one or more RNA substance and at least one or more RNA stabilizing substance that after dilution is ready for injection upon removal from the vial such as by withdrawal using a syringe and needle. As a non-limiting example, the dilution may occur by adding a solution comprising water to a vial that is partially filled to leave volume for adding and mixing diluting solution. As a non-limiting example, the diluting solution may be 0.9% sodium chloride (normal saline, preservative-free). As a non-liming example, the vial may be a multi-dose vial containing at least two doses or containing at least 5 doses or containing at least 10 doses or containing at least 15 doses. As a non-liming example, the vial may be a multi-dose vial that is at least 2 ml size or that is at least 5 ml size or that is at least 10 ml size or that is at least 20 ml size or that is at least 30 ml size.

As a non-limiting example, a multi-chamber syringe 500, as shown in FIG. 7, with a breakable seal 502 between chambers may have a first mixture 508 including at least one or more RNA stabilizing substance, at least one or more RNA substance, and at least one or more cellular uptake agents, such as lipids, in one chamber 504 and an aqueous solution 510, such as water, in a second chamber 506. At time of use the seal 502 between the chambers 504 and 506 is broken and the contents of both chambers 504 and 506 are mixed to induce at least one or more cellular uptake agent, such as lipids, to at least partially bind to, complex with or encapsulate at least part of one or more RNA substance prior to use. For example, the seal 502 may be broken by advancing or turning a plunger assembly 512 or portion thereof to puncture a membrane or otherwise enable mixing. In another non-limiting example, at least one or more RNA substance and at least one or more RNA stabilizing substance are in one chamber and both an aqueous solution, such as water, and at least one or more cellular uptake agent, such as a polymer, are in a second chamber with a breakable seal between the chambers. At time of use the seal is broken and the contents of the two chambers are mixed to induce at least one or more cellular uptake agent, such as a polymer, to at least partially bind to, complex with or encapsulate at least part of one or more RNA substance prior to use.

The containers that may hold a combination comprising at least one or more RNA stabilizing substance and at least one or more RNA substance may be made of any materials suitable for storing and shipping at least one RNA stabilizing substance and at least one RNA substance including, but not limited to, glass, metal, ceramic, plastic or other polymeric material that does not degrade or modify the container's contents or be degraded or modified by the container's contents. The container may have an access port that is penetrated or removed to access the interior of the container including accessing at least part of the contents of the container. The containers may have an access port, such as a screw lid, removable tab, resealable plug or cap or closure that may be penetrated, such as by a hollow tube, such as a hollow needle, to access the interior of the container either to add one or more materials to the contents of the container or to remove at least part of the contents from the interior of the container.

The containers may be used to add materials to the at least one or more RNA stabilizing substance and at least one or more RNA substance. As a non-limiting example, a syringe containing a substance comprising at least one or more lipid may have at least part of its contents transferred to the interior of the container through the access port such as by using a hollow needle penetrating a resealing closure. As another non-limiting example, the syringe may contain an aqueous solution, such as water, that is at least partially transferred to a container having contents comprising at least one RNA stabilizing substance and at least one RNA substance.

The containers, described above, may also hold a combination comprising at least one or more RNA stabilizing substance, at least one or more RNA substance, and at least one or more cellular uptake agent. The containers, described above, may be made of any materials suitable for storing and shipping at least one RNA stabilizing substance, at least one RNA substance and at least one cellular uptake agent including, but not limited to, glass, metal, ceramic, plastic or other polymeric material that does not degrade or modify the container's contents or be degraded or modified by the container's contents.

The containers, described above, may be used to add materials to the at least one or more RNA stabilizing substance, at least one or more RNA substance, and at least one or more cellular uptake agent. As another non-limiting example, the syringe may contain an aqueous solution, such as water, that is at least partially transferred to a container having contents comprising at least one RNA stabilizing substance and at least one RNA substance and at least one cellular uptake agent.

### Uses and Applications:

In one method of use a combination comprising at least one or more RNA stabilizing substance and at least one or more RNA substance is a combination stored in a chamber at a temperature greater than or equal to about −60° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about −40° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about −20° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 0° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 10° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 20° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 30° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

Embodiments of the foregoing methods of use include using one or more RNA stabilizing substance mixed with one or more RNA substance.

In one method of use a combination comprising at least one or more RNA stabilizing substance and at least one or more RNA substance is a combination stored at a temperature less than the melting point of the combination of substances.

In one method of use a combination comprising at least one or more RNA stabilizing substance and at least one or more RNA substance is a combination stored at a temperature less than the melting point of the combination of substances.

In one method of use a combination comprising at least one or more RNA stabilizing substance and at least one or more RNA substance and at least one or more cellular uptake agent is a combination stored at a temperature less than the melting point of the combination of substances.

In one method of use a combination comprising at least one or more RNA stabilizing substance and at least one or more RNA substance and at least one or more cellular uptake agent is a combination stored at a temperature less than the melting point of the combination of substances.

### Applications and Methods of Use

One embodiment of the present invention is the method whereby one or more RNA stabilizing substance may be combined, such as by mixing, with at least one or more RNA substance to produce a mixture comprising at least one or more RNA stabilizing substance and at least one or more RNA substance. As a non-limiting example one or more RNA substance may be mixed with one or more RNA stabilizing substance.

Another embodiment of the present invention is the method whereby one or more RNA stabilizing substance may be combined, such as by mixing, with at least one or more RNA substance to produce a mixture comprising at least one or more RNA substance and at least one or more RNA stabilizing substance at one or more of the RNA stabilizing substance concentrations within the RNA stabilizing substance molar concentration list or RNA stabilizing substance weight percent concentration list as described herein. These same methods may be used to combine other substances, including, but not limited to cellular uptake agents, with one or more RNA substances and one or more RNA stabilizing substances to produce a mixture comprising one or more RNA substance, one or more RNA stabilizing substance, and one or more cellular uptake agent.

One embodiment of the present invention is the method whereby one or more RNA stabilizing substance may be combined, such as by mixing, with at least one or more RNA substance to produce a composition comprising at least one or more RNA substance and at least one or more RNA stabilizing substance. Another embodiment of the present invention is the method whereby one or more RNA stabilizing substance may be combined, such as by mixing, with one or more RNA substance to produce a composition comprising at least one or more RNA substance and at least one or more RNA stabilizing substance at one or more of the RNA stabilizing substance concentrations within the RNA stabilizing substance molar concentration list or RNA stabilizing substance weight percent concentration list as described herein. These same methods may be used to combine other substances, including, but not limited to cellular uptake agents, with one or more RNA substances and one or more RNA stabilizing substances to produce a composition comprising one or more RNA substance, one or more RNA stabilizing substance, and one or more cellular uptake agent.

One embodiment of the present invention is the method whereby one or more RNA stabilizing substance may be combined with one or more RNA substance such as by, including but not limited to, mixing, pipetting, blending, stirring, inverting, submerging, vortexing, shaking, lyophilizing, vaporizing, or sublimating such that at least one or more RNA stabilizing substance is at least intimately associated with or partially contacting or at least partially encapsulating at least one or more RNA substance.

One embodiment of the present invention is the method whereby one or more RNA stabilizing substance may be combined with one or more RNA substance by mixing the substances using known methods. These methods include, but are not limited to, stirring, fluid flow agitation, vortexing, inverting, pipetting, blending, multiple channel fluidics, low shear blending, microfluidic mixing, or using static mixers. These same methods may be used to combine other substances, including, but not limited to cellular uptake agents, with one or more RNA substances and one or more RNA stabilizing substances.

Embodiments of methods described herein may be independent of the order in which each substance may be combined or mixed together. As a non-limiting example one or more RNA stabilizing substance may be combined with one or more RNA substance or one or more RNA substance may be combined with one or more RNA stabilizing substance by the same method.

One embodiment of the present invention is the method whereby one or more RNA stabilizing substances may be combined with at least one or more RNA substance in a container comprising glass, plastic, ceramic, an elastomer, a polymer, or metal.

One embodiment of the present invention is the method whereby one or more RNA stabilizing substances may be combined with at least one or more RNA substance and introduced into a container comprising glass, plastic, ceramic, an elastomer, a polymer, or metal.

In one embodiment, single doses of a stabilized RNA composition may be packaged and sealed. In one embodiment, multiple doses of a stabilized RNA composition may be packaged and sealed in one packaging unit. As a non-limiting example, single doses or multiple doses may be packaged in chambers.

In another embodiment of the present invention at least one or more RNA stabilizing substance may also be used in conjunction with lyophilization of at least one or more RNA substance.

In a further aspect, the present invention further provides the use of the inventive method in the manufacture of a medicament, a therapeutic, or a vaccine.

In one embodiment of the present invention, one or more RNA stabilizing compositions may be used in the manufacture of a medicament, a therapeutic, or a vaccine.

As used herein, stabilized RNA compositions or RNA stabilizing compositions, may be any composition, combination, or mixture comprising at least one or more RNA stabilizing substance and at least one or more RNA substance as described herein. Stabilized RNA compositions or RNA stabilizing compositions may also comprise one or more cellular uptake agents.

According to yet another aspect of the present invention, a pharmaceutical composition is provided, wherein a pharmaceutical composition may comprise one or more RNA stabilizing compositions as described herein.

In one embodiment a pharmaceutical composition may comprise one or more composition, combination, or mixture comprising at least one or more RNA stabilizing substance and at least one or more RNA substance as described herein. In another embodiment a pharmaceutical composition may comprise one or more composition, combination, or mixture comprising at least one or more RNA stabilizing substance, at least one or more RNA substance, and at least one or more cellular uptake agent as described herein.

In another embodiment, a pharmaceutical composition may comprise one or more additional pharmaceutically acceptable ingredient, such as a pharmaceutically acceptable carrier or vehicle.

In another embodiment one or more RNA substance within a pharmaceutical composition may comprise at least one or more pharmaceutically active ingredients.

In one embodiment a pharmaceutical composition may comprise one or more pharmaceutically active RNA component.

In another embodiment a pharmaceutical composition may comprise one or more non-RNA pharmaceutically active component. Wherein a non-RNA pharmaceutically active component may be a compound that has a therapeutic effect against a particular medical indication, such as, but not limited to, cancer diseases, autoimmune disease, allergies, infectious diseases or a further disease, as non-limiting examples. Non-limiting examples of such compounds may include, but are not limited to: peptides or proteins, (therapeutically active) low molecular weight organic or inorganic compounds (molecular weight less than 5,000, preferably less than 1,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In one embodiment one or more pharmaceutical compositions, as described herein, may be administered orally, sublingually, transdermally, ophthalmically, parenterally, subcutaneous, intravenous, intramuscular, by inhalation, topically, rectally, nasally, buccally, vaginally or via an implanted reservoir. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In one method of use one or more pharmaceutical compositions, as described herein, may be administered orally, sublingually, transdermally, ophthalmically, parenterally, subcutaneous, intravenous, intramuscular, by inhalation, topically, rectally, nasally, buccally, vaginally or via an implanted reservoir. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In one method of use one or more RNA stabilizing composition as described herein may be used to produce a medicament, vaccine, or therapeutic agent comprising at least one or more RNA substance and at least one or more RNA stabilizing substance.

In one method of use one or more RNA stabilizing composition as described herein may be used to produce a medicament, vaccine, or therapeutic agent comprising at least one or more RNA substance, at least one or more RNA stabilizing substance, and at least one or more cellular uptake agent.

In one method of use one or more RNA stabilizing composition as described herein may be combined with a medicament, vaccine, or therapeutic agent comprising at least one or more RNA substance and at least one or more RNA stabilizing substance.

In one method of use one or more RNA stabilizing composition as described herein may be combined with a medicament, vaccine, or therapeutic agent comprising at least one or more RNA substance, at least one or more RNA stabilizing substance, and at least one or more cellular uptake agent.

In another method of use one or more RNA stabilizing composition as described herein may be used for one or more of the following applications, including, but not limited to, treating a disease, preventing a disease, or producing a cellular response in one or more of the following organisms or cells, which may include but are not limited to: humans, primates, animals, vertebrate animals, eukaryotic cells, eukaryotes, protozoa, prokaryotic cells, plant cells, plants, fungal cells, fungi, insect cells, insects, bacterial cells, bacteria, mycoplasma, protozoa, plasmodium, or mammalian cells, including but not limited to the cells of primates, animals, vertebrate animals, and the cells of humans.

In one method of use one or more RNA stabilizing composition as described herein may be used for in vivo, in vitro, in situ, or ex vivo applications. Such applications may include, but are not limited to, one or more of the following: a laboratory reagent, research applications, agricultural applications, agricultural treatment, veterinary applications, animal treatment, pharmaceutical applications, human treatment, soil treatment, pesticide, herbicide, plant application, plant treatment, antibody production, protein expression applications, insect treatment, vaccine production, therapeutic agent production, drug production, or medicament production, or combinations thereof.

In another method of use one or more RNA stabilizing composition as described herein may be used for one or more of the following applications, including but not limited: syringe, prefilled syringe, injection, nasal spray, transdermal patch, eye drop, oral spray, aerosol, inhaler, nebulizer, oral tablet, pill, sublingual tablet, sublingual drop, suppository, mucosal spray, cream, lozenge, lotion, balm, syrup, or ointment.

In another method of use one or more RNA stabilizing composition as described herein may be used in composition that also comprises a cellular uptake agent and used as a mucosal spray. Wherein, a mucosal spray may be any container that can be squeezed, pressurized, or applied in such a manner to aerosolize, spray, mist, aspirate, drop, squirt, apply, administer, or direct a combination comprising at least one RNA stabilizing substance and at least one or more RNA substance combined in a mixture comprising at least one cellular uptake agent into or onto a mucosal surface such as a nasal passage, airway, throat, lung, eye, or other mucosal surface within a human, primate, animal, or vertebrate animal.

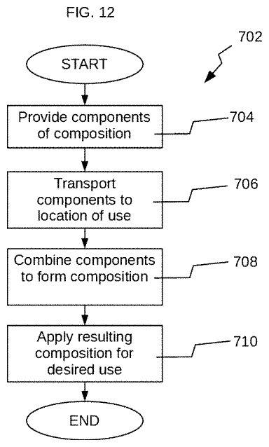

FIG. 12 is a flowchart that summarizes a process 702 for producing and using an RNA product in accordance with the present invention. The process 702 is initiated by providing (704) components of the RNA product or composition. The components may be obtained separately or may be provided as part of a kit or in a preloaded, multi-chamber syringe as described above, among other possibilities. Moreover, the composition, components, or combinations of components, may be provided in bottles, containers, vials, tubes, syringes, blisters, capsules, cartridges, or other packaging methods. The components may then be stored at the location of use or transported (706) to the location of use. Depending on the specific implementation, the components or some of the components may be refrigerated, frozen, or otherwise maintained in a temperature-controlled environment during transportation and storage.

When ready for use, the components can be combined (708) to yield the desired composition. For example, the individual components may be combined in a bottle, vial, or other container by adding the individual components to the container, e.g., by pouring, using a pipette, syringe, or the like, by adding lyophilized pellets, by measuring powders, or by any other suitable method. In certain implementations, the components may be mixed by breaking a breakable seal of a multi-compartment container such as a multi-chamber syringe. Finally, the resulting composition may be applied (710) for the desired use. As otherwise noted herein, the stabilized RNA products of the present invention may be utilized in a variety of fields such as therapeutics, diagnostics, reagents, agriculture, or biological assays. In addition, the product may be packaged and distributed as a medicament, a therapeutic, or vaccine. Moreover, in the case of pharmaceutical compositions, the product may be packaged and distributed for administration orally, sublingually, transdermally, ophthalmically, parenterally, subcutaneously, intravenously, intramuscularly, by inhalation, topically, rectally, nasally, buccally, vaginally, or via an implanted reservoir as otherwise described herein. The RNA product may be used for a variety of applications including treating a disease, preventing a disease, or producing a cellular response as otherwise described herein. The product may be used in in vivo, in vitro, in situ, or ex vivo applications. Such applications may include a laboratory reagent, research applications, agricultural applications, agricultural treatment, veterinary applications, animal treatment, pharmaceutical applications, human treatment, soil treatment, pesticide, herbicide, plant application, plant treatment, antibody production, protein expression applications, insect treatment, vaccine production, therapeutic agent production, drug production, medicament production, or combinations thereof. Accordingly, applying the resulting composition for the desired use will vary depending on the nature of the composition and the intended use among other things.

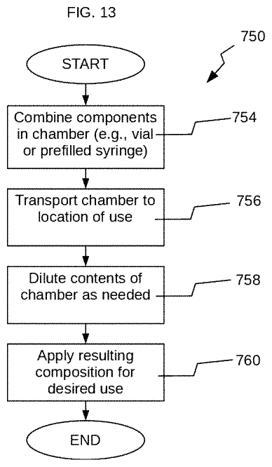

FIG. 13 is a flowchart that summarizes a process 750 for producing and using an RNA product in accordance with the present invention. The process 750 is initiated by providing (754) components of the RNA product or composition and combining them in a chamber or combining them and adding the combination to a chamber. The chamber may be any suitable chamber and may be, as non-limiting examples, a single use or multiuse vial. Moreover, the chamber may be, as non-limiting examples, bottles, containers, vials, tubes, syringes (including prefilled or single use syringes), blisters, capsules, cartridges, or other packaging. The chamber with components may then be stored at the location of use or transported (756) to the location of use. Depending on the specific implementation, the chambers, holding components, may be refrigerated, frozen, or otherwise maintained in a temperature-controlled environment during transportation and storage.

When ready for use, the components in the chamber may be combined with one or more diluents (758) to yield the desired concentration for final use. For example, the chambers produced at step (754) may, for example, contain concentrated mixture needing dilution or solids needing to be dissolved, for example in a bottle, vial, syringe, or other container. Alternatively, when ready for use, if the components introduced into the chamber are such that no dilution is needed then the contents are not diluted. Alternatively, part or all of the contents of the chamber may be withdrawn and added to another container, a non-limiting example being a bag containing an IV solution. At this stage other materials may be added.

Finally, the resulting composition may be applied (760) for the desired use. As otherwise noted herein, the stabilized RNA products of the present invention may be utilized in a variety of fields such as therapeutics, diagnostics, reagents, agriculture, or biological assays. In addition, the product may be packaged and distributed as a medicament, a therapeutic, or vaccine. Moreover, in the case of pharmaceutical compositions, the product may be packaged and distributed for administration orally, sublingually, transdermally, ophthalmically, parenterally, subcutaneously, intravenously, intramuscularly, by inhalation, topically, rectally, nasally, buccally, vaginally, or via an implanted reservoir as otherwise described herein. The RNA product may be used for a variety of applications including treating a disease, preventing a disease, or producing a cellular response as otherwise described herein. The product may be used in in vivo, in vitro, in situ, or ex vivo applications. Such applications may include a laboratory reagent, research applications, agricultural applications, agricultural treatment, veterinary applications, animal treatment, pharmaceutical applications, human treatment, soil treatment, pesticide, herbicide, plant application, plant treatment, antibody production, protein expression applications, insect treatment, vaccine production, therapeutic agent production, drug production, medicament production, or combinations thereof. Accordingly, applying the resulting composition for the desired use will vary depending on the nature of the composition and the intended use among other things. As a non-limiting example, a chamber may be at least partially filled with components comprising one or more RNA substance and one or more RNA stabilizing substance followed by the chamber being prepared for shipping and storage (as a non-limiting example by undergoing steps comprising being packaged or placed in a shipping and storage container) followed by the chamber being transported to the location use, then removed from packaging and prepared for use by adding one or more diluents to the chamber and mixing, and with the appropriate amount of diluted mixture withdrawn from the chamber and administered to a patient as a therapeutic agent.

### EXAMPLES

The following non-limiting examples describe examples of the invention in more detail and in no way are to be construed as limiting the scope thereof.

### Example 1

In Vitro Transcription

The RNA was synthesized by in vitro transcription from a linear DNA construct with an upstream T7 RNA Polymerase promoter followed by the coding sequence for gene of interest. In vitro transcription was performed using the HiScribe T7 Quick High Yield RNA Synthesis Kit (New England Biolabs, Ipswich, Mass.) according to the manufacturer's directions. Briefly, 2.5 μg template DNA was mixed with 25 μL NTP buffer mix and 5 μL T7 RNA polymerase mix. The entire reaction volume was brought to 50 μL with molecular biology grade H₂O and incubated in a thermal cycler at 37° C. for 2 hrs.

Following in vitro transcription, the RNA was purified using a Monarch RNA

Cleanup Kit (New England Biolabs, Ipswich, Mass.) according to the manufacturer's directions. Briefly, 1 spin column was used for each 50 μL reaction. Following binding of the RNA to the spin column, 2 washes of 500 μL were performed and the RNA was eluted with 100 μL of molecular biology grade H₂O. The purified RNA was then stored at −80° C.

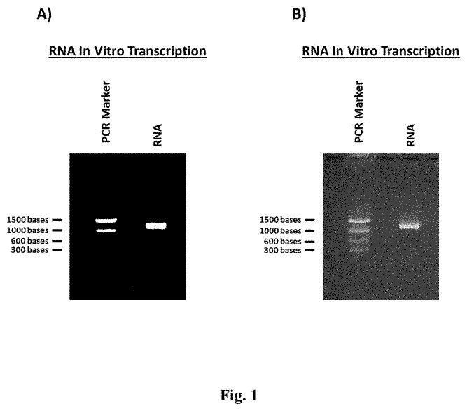

The in vitro transcribed and purified RNA was analyzed by denaturing agarose gel electrophoresis. Briefly, approximately 5 μg RNA was diluted 1:2 with 2×RNA loading dye (New England Biolabs, Ipswich, Mass.) and heated to approximately 65-70° C. for 2 minutes to denature the RNA. The final concentration of RNA and loading dye was approximately: 5 μg RNA, 47.5% formamide, 0.01% SDS, 0.01% bromophenol blue, 0.005% xylene cyanol and 0.5 mM EDTA. The RNA was run on a 1.5% agarose gel in 1× Tris-Acetate EDTA buffer (Tris 40 mM, Acetate 20 mM, EDTA 1 mM, pH 8.0) supplemented with 0.06% sodium hypochlorite (NaClO) to prevent renaturing and degradation of the RNA during electrophoresis. The running buffer also contained 1×TAE buffer supplemented with 0.06% sodium hypochlorite. RNA was visualized using SmartGlow fluorescent nucleic acid prestain (Accuris Instruments, Edison, N.J.) according to the manufacturer's directions. Denaturing agarose gel electrophoresis was carried out for 1 hr at 80 v. The in vitro transcribed and purified RNA analyzed by denaturing agarose gel electrophoresis is shown in FIG. 1.

RNA concentration was measured by absorbance of the purified RNA at 260 nm using a Nanodrop ND-1000 (Thermo Fisher Scientific, Waltham, Mass.). Typical RNA concentration following in vitro transcription and purification was approximately 4 mg/mL to 5 mg/mL.

### Example 2

Stability of RNA at Various Temperatures

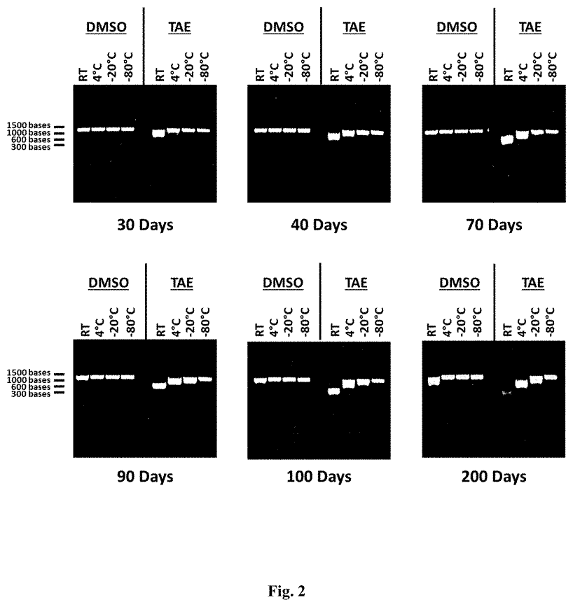

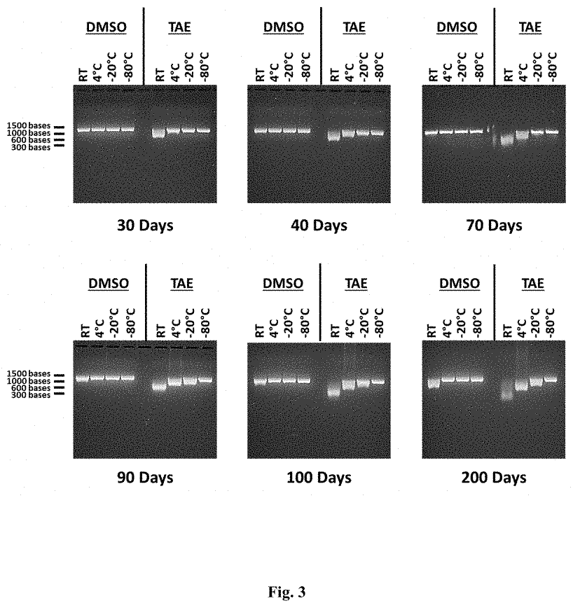

In vitro transcribed RNA was diluted approximately at a ratio of 1:10 (approximately 450 μg/mL) in either DMSO or 1× Tris-Acetate EDTA buffer (TAE). The final concentration of DMSO was approximately 90% DMSO. The final concentration of TAE was approximately Tris 40 mM, Acetate 20 mM, EDTA 1 mM, pH 8.0. Following dilution of the RNA in either DMSO or TAE, the samples were then stored at 4 different temperatures: room temperature (approximately 20-25° C.), approximately 4° C., approximately −20° C., and approximately −80° C. Samples were then analyzed by denaturing agarose gel electrophoresis as described above at selected timepoints to measure RNA degradation and the stability of the RNA samples stored in either DMSO or TAE. During storage, 10 μL of each sample was analyzed at selected timepoints by agarose gel electrophoresis to measure RNA degradation and the stability of the RNA samples stored at each temperature in either DMSO or TAE. FIG. 2 and FIG. 3 show the agarose gel electrophoresis of each RNA sample following storage of the RNA for approximately 30 days to 200 days at various temperatures.

The RNA sample stored in DMSO displays increased stability and reduced rate of degradation of the RNA sample as shown by agarose gel electrophoresis. The RNA sample stored in TAE begins to show notable degradation at room temperature after approximately 30 days as indicated by the smearing of the RNA band, decreased fluorescence intensity, and lower apparent molecular weight compared to the −80° C. RNA sample and initial in vitro transcribed RNA sample. While the RNA sample stored in DMSO does not show notable signs of degradation until approximately 90 days at room temperature. Furthermore, the RNA sample stored in TAE begins to show notable signs of degradation following approximately 40 days at 4° C. While the RNA sample stored in DMSO does not show notable signs of degradation following approximately 200 days at 4° C. In addition, the RNA sample stored in TAE begins to show notable signs of degradation following approximately 90 days at −20° C. While the RNA sample stored in DMSO does not show notable signs of degradation following 200 days at −20° C. Following 90 days, it becomes apparent that the RNA sample stored in DMSO shows comparable and/or better stability at room temperature when compared to the RNA sample stored in TAE at 4° C. or −20° C. Furthermore, RNA stored in DMSO at 4° C. still displays a measurable band of comparable size and fluorescence intensity compared to the −80° C. RNA sample and initial in vitro transcribed RNA sample following 200 days, while the RNA stored in TAE shows little to no band of comparable size and fluorescence intensity compared to the −80° C. RNA sample and initial in vitro transcribed RNA sample following 200 days at 4° C.

### Example 3

Accelerated RNA Stability Testing at 60° C.

In vitro transcribed RNA was diluted approximately at a ratio of 1:10 (approximately 450 μg/mL) in different compositions containing either sodium acetate buffer (pH 5.2), or a mixture of DMSO and sodium acetate buffer (pH 5.2) as follows:

1. 50 mM Sodium Acetate, pH 5.2

2. 90% DMSO+50 mM Sodium Acetate, pH 5.2

3. 80% DMSO+50 mM Sodium Acetate, pH 5.2

4. 70% DMSO+50 mM Sodium Acetate, pH 5.2

5. 60% DMSO+50 mM Sodium Acetate, pH 5.2

6. 50% DMSO+50 mM Sodium Acetate, pH 5.2

7. 40% DMSO+50 mM Sodium Acetate, pH 5.2

8. 30% DMSO+50 mM Sodium Acetate, pH 5.2

9. −80° C.

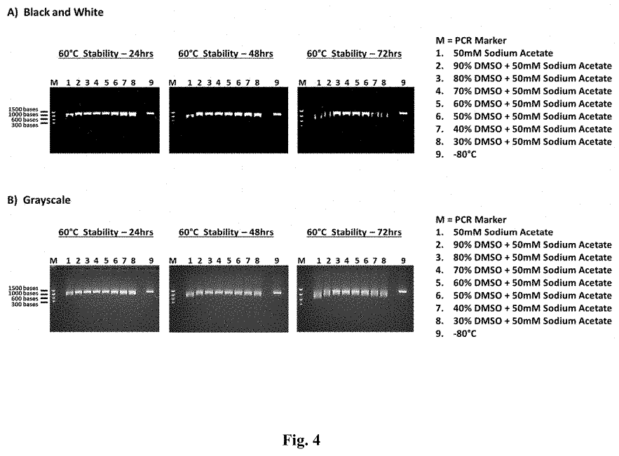

Following dilution of each sample in each respective RNA storage environment, samples were stored at approximately 60° C. for up to 72 hours. During storage at 60° C., 10 μL of each sample was analyzed at selected timepoints by agarose gel electrophoresis to measure RNA degradation and the stability of the RNA samples in each RNA storage environment. FIG. 4 shows the agarose gel electrophoresis of each RNA sample following storage of the RNA for approximately 24 hours to 72 hours at 60° C. Following 24 hours at 60° C. samples 3-5 display little to no RNA degradation as indicated by the bands of comparable size and fluorescent intensity as compared to the −80° C. reference sample. Following 48 hours at 60° C. the increased stability of samples 3-5 becomes more apparent. Samples 3-5 display bands of comparable size and fluorescent intensity with minimal RNA degradation as compared to the −80° C. reference sample. Meanwhile, the remaining samples show varying degrees of RNA degradation as indicated by the broadening of the RNA bands, decreased fluorescence intensity, and lower apparent molecular weight. Following 72 hours at 60° C. samples 3-5 display increased RNA stability with significantly less RNA degradation as compared to the other samples. Sample 9 is a purified RNA sample stored at −80° C. for stability comparison.

### Example 4

Accelerated RNA Stability Testing at 60° C.

In vitro transcribed RNA was diluted approximately at a ratio of 1:10 (approximately 450 μg/mL) in different compositions containing either sodium acetate buffer (pH 5.2), DMSO, or acetylcholine chloride as follows:
1. 50 mM Sodium Acetate, pH 5.2
2. 70% DMSO+50 mM Sodium Acetate, pH 5.2
3. 70% DMSO+500 mM Acetylcholine Chloride+50 mM Sodium Acetate, pH 5.2

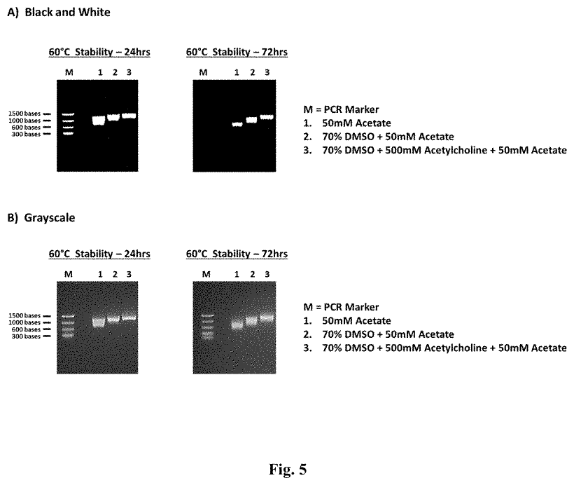

Following dilution of each sample in each respective RNA storage environment, samples were stored at approximately 60° C. for up to 72 hours. During storage at 60° C., 10 μL of each sample was analyzed at selected timepoints by agarose gel electrophoresis to measure RNA degradation and the stability of the RNA samples in each RNA storage environment. FIG. 5 shows the agarose gel electrophoresis of each RNA sample following storage of the RNA for approximately 24 hours to 72 hours at 60° C. Following 24 hours at 60° C. sample 2 displays minor RNA degradation, while sample 3 displays essentially no RNA degradation as indicated by the relatively sharp band with comparable apparent molecular weight and fluorescence intensity to the original in vitro transcribed RNA. Meanwhile, sample 1 displays significant RNA degradation as indicated by the broadening of the RNA band, decreased fluorescence intensity, and lower apparent molecular weight compared to sample 2 and sample 3. Following 72 hours at 60° C. the increased RNA stability of sample 3 becomes more apparent. Sample 3 displays some minor RNA degradation but when compared to samples 1 and 2, sample 3 maintains a relatively sharp band with comparable apparent molecular weight to the original in vitro transcribed RNA.

### Example 5

Accelerated RNA Stability Testing at 60° C.

In vitro transcribed RNA was diluted approximately at a ratio of 1:10 (approximately 450 μg/mL) in different compositions containing either sodium acetate buffer (pH 5.2), DMSO, or acetylcholine chloride as follows:
1. 50 mM Sodium Acetate, pH 5.2
2. 70% DMSO+50 mM Sodium Acetate, pH 5.2
3. 70% DMSO+1M Acetylcholine Chloride+50 mM Sodium Acetate, pH 5.2
4. 70% DMSO+500 mM Acetylcholine Chloride+50 mM Sodium Acetate, pH 5.2

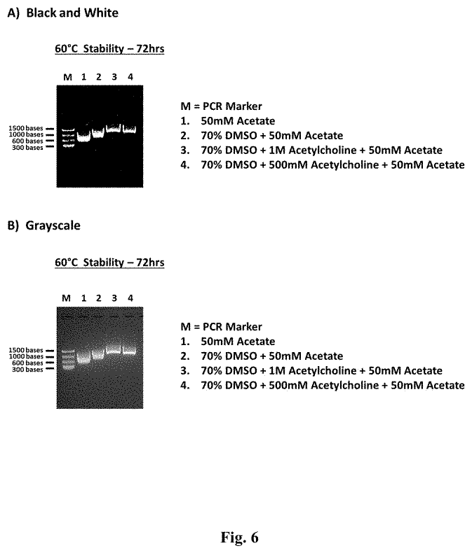

Following dilution of each sample in each respective RNA storage environment, samples were stored at approximately 60° C. for up to 72 hours. During storage at 60° C., 10 μL of each sample was analyzed at selected timepoints by agarose gel electrophoresis to measure RNA degradation and the stability of the RNA samples in each RNA storage environment. FIG. 6 shows the agarose gel electrophoresis of each RNA sample following storage of the RNA for approximately 72 hours at 60° C. Following 72 hours at 60° C. samples 3 and 4 display increased RNA stability as compared to samples 1 and 2. Samples 3 and 4 display little to no RNA degradation following 72 hrs at 60° C. as indicated by the relatively sharp bands with comparable apparent molecular weight and fluorescence intensity to the original in vitro transcribed RNA.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed:

1. A composition comprising at least one RNA substance and at least one RNA stabilizing substance.

2. The composition of claim 1, wherein the at least one RNA stabilizing substance comprises at least one aprotic substance.

3. The composition of claim 2, wherein the total weight percentage of all aprotic substances in the composition is at least 0.1 percent or one (1) nanomolar.

4. The composition of claim 1, further comprising one or more of a cellular uptake agent, a chelating agent, a buffering agent, a salt, and a solvent.

5. The composition of claim 2, further comprising one or more of a cellular uptake agent, a chelating agent, a buffering agent, a salt, and a solvent.

6. The composition of claim 2, wherein the at least one aprotic substance comprises one or more of DMSO and acetylcholine.

7. The composition of claim 2, wherein the at least one aprotic substance comprises one or more of DMSO, dimethyl sulfone, triacetin, diethyl carbonate, and diethyl sulfoxide.

8. The composition of claim 2, wherein the at least one aprotic substance is aprotic in a mixture at a physiologic pH.

9. The composition of claim 2, wherein the at least one aprotic substance comprises a choline-based ester.

10. The composition in claim 9, wherein the at least one choline-based ester comprises one or more of acetylcholine, butyrylcholine, and methacholine.

11. The composition of claim 5, wherein said composition includes one or more cellular uptake agents comprising at least one polymer.

12. The composition of claim 11, wherein the at least one polymer comprises one or more of a cationic polymer, a polycationic polymer, an ionizable polymer, and a zwitterionic polymer.

13. The composition of claim 5, wherein the composition includes one or more cellular uptake agents comprising at least one lipid.

14. The composition of claim 13, wherein the at least one lipid comprises one or more of a cationic lipid, an ionizable lipid, a zwitterionic lipid, a PEG modified lipid, PEG conjugated lipid, hydrophilic polymer modified lipid, and a hydrophilic polymer conjugated lipid.

15. The composition of claim 5, wherein the composition includes at least one cellular uptake agent comprising at least one detergent.

16. The composition of claim 15, wherein the at least one detergent comprises one or more of a cationic detergent, a non-ionic detergent, an ionizable detergent, and a zwitterionic detergent.

17. The composition of claim 5, wherein the composition includes at least one solvent comprising water.

18. The composition of claim 1, wherein the at least one RNA substance comprises a coding RNA.

19. The composition of claim 18, wherein the coding RNA comprises one or more of mRNA and self-amplifying RNA.

20. The composition of claim 1, wherein the at least one RNA substance comprises a non-coding RNA.

21. The composition of claim 20, wherein the non-coding RNA comprises one or more of small interfering RNA (siRNA), microRNA, CRISPR RNA, antisense RNA (asRNA), small activating RNA, and RNA enzyme.

22. The composition of claim 1, wherein the composition comprises a pharmaceutical composition.

23. The composition of claim 22, wherein said pharmaceutical composition comprises one of a medicament, a therapeutic, and a vaccine.

24. The composition of claim 1, wherein said RNA stabilizing substance is effective to maintain a defined level of stability of RNA molecules in said RNA composition when subjected to a defined environment for a defined time period, wherein said defined environment includes temperatures in excess of 0° C., said time period is at least 30 days, and said defined level of stability is defined by degradation of no more than about 50% of said RNA molecules.

25. The method of claim 24, wherein said RNA composition is continuously subjected to said defined environment.

26. The method of claim 24, wherein said defined environment includes temperatures in excess of 10° C.

27. The method of claim 24, wherein said defined environment includes temperatures in excess of 20° C.

28. The method of claim 24, wherein said defined time period is at least 60 days.

29. The method of claim 24, wherein said defined time period is at least 90 days.

30. The method of claim 24, wherein said defined level of stability is defined by degradation of no more than 30% of said RNA molecules.

31. The method of claim 24, wherein said defined level stability is defined by degradation of no more than 10% of said RNA molecules.

32-103. (canceled)
